(19) United States
(12) Patent Application Publication
(10) Pub. No.: US 2010/0105640 A1
(43) Pub. Date: Apr. 29, 2010

(54) Aromatase Inhibitors for Emergency Contraception

(76) Inventors: Robert F. Casper, Toronto (CA); Mohammad Mitwally, Mississausa (CA); Roger Allen Pierson, Saskatchewan (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/989,787

(22) PCT Filed: Jun. 28, 2006
(86) PCT No.: PCT/CA2006/001079

§ 371 Date: Dec. 18, 2009

Related U.S. Application Data
(60) Provisional application No. 60/694,237, filed on Jun. 28, 2005.

(51) Int. Cl. A61K 31/566; A61P 15/04

(52) U.S. Cl. 514/170; 514/171

(57) ABSTRACT

There is disclosed an emergency contraceptive preparation which comprises at least one aromatase inhibitor wherein the preparation comprises at least one dose for administration on one or more days to a female patient following an unprotected sexual encounter until the establishment or continuation of pregnancy of the patient is prevented. Also disclosed is a method of emergency contraception.

### FIELD OF THE INVENTION

This invention relates to a method for preventing unwanted pregnancy in females exposed to unprotected sexual encounter(s) that may lead to pregnancy. The invention involves administration of an aromatase inhibitor (AI) after unprotected sexual exposure. Also disclosed are pharmaceutical preparations and related uses.

### BACKGROUND OF THE INVENTION

Definition of Emergency Contraception

Emergency contraception is used to prevent pregnancy after a coital act not adequately protected by a regular method of contraception. In contrast to early medical abortion, emergency contraception prevents a pregnancy from starting and does not disrupt an established pregnancy⁽¹⁾.

Situations in which Emergency Contraception is Needed

Emergency contraceptives are methods of preventing pregnancy after unprotected sexual intercourse. It is important to stress that they do not protect against sexually transmitted diseases. Emergency contraception can be used when a condom breaks, after a sexual assault, or any time unprotected sexual intercourse occurs

There are four major situations during which emergency contraception is needed after sexual encounter that can result in pregnancy:

1. Lack of contraceptive use during the unprotected sexual encounter

2. Inadequate or inappropriate use of contraceptive method e.g.
Mechanical failure e.g. breakage, slippage or leakage of male or female condom, diaphragm or cervical cap
Failure of spermicide tablet or film to melt before intercourse
Error in practicing coitus interruptus
Missing contraceptive pills
Complete or partial expulsion of intrauterine device
Late injection of depot contraception

3. Exposure to a potential teratogen while not using an effective method of contraception

4. Rape

Currently Available Emergency Contraception Methods

Currently available emergency contraceptives include contraceptive pills and intrauterine devices. There are two types of emergency contraceptive pills. One type uses hormones that are the same type and dose as hormones used in some kinds of ordinary birth control pills (combined estrogen and progesterone). One brand name, approved by the FDA, called “Preven®” (Gynétics, Inc.) is especially packaged and labeled for emergency use⁽²⁾. However, several other brands packaged for ongoing contraception can be used as well. The pills are administered according to the Yuzpe regimen i.e. two doses given 12 hours apart. Each dose contains 0.5 mg of levonorgestrel and 100 μg of ethinyl estradiol. The other type of emergency contraceptive pill contains only progestin. This FDA-approved type is specially packaged and labeled for use as the brand name plan B. The regimen consists of two doses, given 12 hours apart. Each dose contains 0.75 mg of levonorgestrel⁽³⁾.

A third method that is not approved in the US involves orally administered mifepristone, an antiprogestin. Randomized, controlled trials have shown that a single oral 600-mg dose of mifepristone was more effective and had fewer side effects than the Yuzpe regimen⁽⁴⁾.

Success Rate

Data on efficacy are most extensive for the combined estrogen-progestin regimen. A meta-analysis of eight studies including more than 3000 women in total concluded that when used within 72 hours after sex, the combined regimen prevents about 74% of expected pregnancies. The figures from the eight individual studies, however, ranged widely (56% to 89%)⁽⁵⁾.

The largest study of the progestin-only regimen was a randomized trial conducted by the World Health Organization that included 1001 women using the regimen at 21 centers in 14 countries⁽⁶⁾. When used within 72 hours after intercourse, the estimated effectiveness in preventing pregnancy was 85% after levonorgestrel therapy. A smaller studym in which the regimen was used within 48 hours after intercourse found an effectiveness of 60% in pregnancy prevention.

Some data indicates that emergency contraceptive pills are more effective the sooner after intercourse they are taken. In the World Health Organization trial, for example, the prevented pregnancy fraction was 77% if the Yuzpe regimen was used on the first day after intercourse but only 31% if it was used on the third day⁽⁶⁾. The levonorgestrel regimen also showed a decrease in effectiveness with time). However, data also indicate that emergency contraceptive pills retain substantial effectiveness when used more than 72 hours after intercourse^{(9, 10)}. One randomized, controlled trial⁽¹¹⁾ and another cohort study⁽¹²⁾ suggest that emergency contraception confers protection up to 120 hours after coitus.

Mechanism of Action

The mechanisms of action of the various forms of emergency contraception have not been extensively studied and, consequently, are not well understood. However, like all hormonal contraceptives, emergency contraceptive pills probably work through multiple mechanisms that may depend on the timing of their administration in the menstrual cycle. The mechanism of action in any specific case is impossible to determine. When taken before ovulation, emergency contraceptive pills may inhibit ovulation in some women⁽¹³⁻¹⁸⁾. Several studies have shown histological or biochemical alterations in the endometrium after treatment with this regimen, suggesting that it may impair endometrial receptivity to implantation of a fertilized egg^{(17, 19-22)}. However, other studies^{(16, 23, 24)} have found no such endometrial effects. Whether the endometrial changes that have been observed would be sufficient to inhibit implantation remains unclear. Additional mechanisms proposed for medical emergency contraception regimens include changes to the cervical mucus that result in trapping of sperm; alterations in the transport of sperm, egg, or embryo through the reproductive tract^{(25, 26)}; interference with corpus luteum function^{(17, 27)}; and direct inhibition of fertilization. Statistical evidence indicates that current emergency oral contraceptive regimens could not be as effective as data show unless they work by a mechanism of action other than prevention of ovulation⁽²⁸⁾. Intrauterine devices used for emergency contraception may work by any of the mechanisms that account for their effectiveness when used for conventional contraception.

Side Effects of Emergency Contraceptives

In the largest study of the two FDA-approved emergency contraception regimens reported to date, the most commonly reported complaints were nausea, breast tenderness, lower abdominal pain, fatigue, headache, heavier or lighter menstrual bleeding, dizziness, vomiting, and diarrhea (table 1)⁽²⁹⁾. The few case reports of serious adverse events in emergency contraceptive pill users do not support a causal association.

TABLE 1
Side Effects Associated With Two Emergency
Contraceptive Pills Regimens

| | Percent with symptom | | |
|---|---|---|---|
| | (95% confidence interval) | | |
| | Yuzpe | Levonorgestrel | |
| Symptom | (n = 979) | (n = 977) | |
| Nausea | 50.5 (47.3, 53.6) | 23.1 | (20.5, 25.9) |
| Vomiting | 18.8 (16.4, 21.4) | 5.6 | (4.3, 7.3) |
| Dizziness | 16.7 (14.4, 19.1) | 11.2 | (9.3, 13.3) |
| Fatigue | 28.5 (25.7, 31.4) | 16.9 | (14.6, 19.4) |
| Headache | 20.2 (17.8, 22.9) | 16.8 | (14.5, 19.3) |
| Breast tenderness | 12.1 (10.1, 14.3) | 10.8 | (8.9, 12.9) |
| Low abdominal pain | 20.9 (18.4, 23.6) | 17.6 | (15.3, 20.1) |
| All other adverse effects* | 16.7 (14.4, 19.1) | 13.5 | (11.4, 15.8) |

*Mostly diarrhea and some irregular bleeding or spotting. Source: Reference.⁷

Current Practices of Emergency Contraceptives

Unintended pregnancy is a major public health problem worldwide and especially in the United States. Almost half of all United States women aged 15 to 44 years in 1994 have had at least one unwanted pregnancy at some time in their lives. Almost half of the 5.4 million pregnancies ending in 1994 were unintended and resulted in unwanted or mistimed births or in abortion. If these abortion rates persist, more than 40% of United States women will have had at least one induced abortion by the time they reach menopause⁽³⁰⁾. The consequences of unintended pregnancy can be serious, including maternal death and morbidity, low birth weight babies, birth defects, infant death, maternal and child abuse, and social and economic hardship for all involved⁽³¹⁾.

Emergency contraceptive pills have the potential to reduce the incidence of unintended pregnancy substantially. Most unintended pregnancies occur after an immediately apparent contraceptive failure e.g. a condom breaks, oral contraceptive pills are missed, a spermicidal tablet fails to melt—or after a couple fails to use any contraception at all. At least one of these events has been estimated to occur in more than 64 million menstrual cycles per year in the United States. If emergency contraceptive pills were used in three-quarters of these situations, unintended pregnancies and consequent abortions could be reduced by as much as half⁽³²⁾. Widespread use of emergency contraception could prevent an estimated 1.7 million unintended pregnancies and 800,000 abortions each year^{(33, 34)}.

These benefits can be realized, however, only if women have ready access to the therapy. Both emergency contraception products currently approved by the FDA are labeled for use within the first 72 hours after intercourse. Even within this time period, both regimens appear to be substantially more effective the sooner they are used⁽³⁵⁾. Any delay in treatment reduces efficacy, leading to an increased risk of treatment failure and consequent unintended pregnancy.

As mentioned above the United States has one of the highest rates of abortion of any developed country⁽³⁶⁾. Emergency contraception has proven to be safe and effective⁽³⁷⁻⁴¹⁾ and has the potential to decrease abortion by up to 50%⁽⁴²⁾. However, use remains limited⁽⁴³⁻⁴⁵⁾, despite extensive nationwide public education campaigns⁽⁴⁶⁻⁴⁸⁾ and the introduction of two dedicated emergency contraception products. Knowledge about emergency contraception by both patients and providers remains insufficient^{(43, 44, 49-53)}, and access remains limited because of difficulties obtaining prescriptions⁽⁵⁴⁾ or unavailability of emergency contraception pills at local pharmacies^{(48, 55)}.

Whether emergency contraception can fulfill its potential for decreasing unintended pregnancies depends on women's ability to easily obtain and use it. The majority of US women remain unfamiliar with emergency contraception. Of those reporting any familiarity, fewer than 25% know how to obtain pills and that they must be used within 72 hours of unprotected intercourse^{(43, 44, 47, 49, 56)}. Even those who know how to obtain emergency contraception may not be able to secure a prescription and find a pharmacy that stocks it within 72 hours. The efficacy of emergency contraception is improved the earlier it is used^{(57, 58)}. Therefore, an emergency contraceptive that has a longer window of effectiveness, ideally up to one week after unprotected intercourse would be a major benefit.

Endometrial Physiology

Human endometrium is a unique tissue that undergoes sequential phases of proliferation, and secretory changes followed by tissue shedding and bleeding during menstruation. Tissue remodeling is a distinct feature of human endometrium in the secretory phase that prepares endometrium for implantation during the “receptive phase” of the cycle. If implantation does not occur, this tissue rapidly undergoes dissolution during the menstrual period. The processes of bleeding and tissue shedding during menstruation are precisely controlled by a number of systemic and local factors. The systemic signal that leads to menstruation is the withdrawal of the steroid hormones⁽⁵⁹⁻⁶¹⁾.

Menstruation is the process by which the endometrium is discarded each month if pregnancy fails to occur. It involves sloughing of the endometrium over a period of days, bleeding and subsequent repair so that the uterus is receptive to an implanting embryo in the next cycle. Work carried out in the 1930s by Markee⁽⁶²⁾, Corner and others⁽⁶³⁾ established that ovarian steroids, oestradiol (E2) and progesterone (P), were responsible for the changes in endometrial structure and function throughout the cycle.

Within the uterus, the female sex steroids estrogen and progesterone play pivotal roles in the establishment of a suitable environment for embryo implantation and pregnancy. More specifically, these steroids regulate a multitude of cellular processes, which include cell proliferation and differentiation, as well as regulation of vascular permeability, angiogenesis and adenogenesis. To bring about these changes, estrogen and progesterone must appropriately modulate a variety of factors, which include growth factors, cytokines, extracellular matrix proteins and adhesion molecules⁽⁶⁴⁻⁷⁰⁾.

Steroids interact with their target organs via specific nuclear receptors. The expression of endometrial sex steroid receptors (progesterone receptor (PR), oestrogen receptor (ER), androgen receptor (AR), all of which are nuclear proteins, varies both temporally and spatially across the menstrual cycle⁽⁷¹⁻⁷⁷⁾. The expression of ER and PR are under dual control of E2 and P. Both endometrial ER and PR are up-regulated during the follicular phase by ovarian E2 and subsequently down regulated in the luteal phase by P acting at both the transcriptional and the post-transcriptional levels⁽⁷⁸⁾.

Experiments with rhesus macaques that have been treated with oestrogen and progesterone indicate that the induction of menstruation is identical under the following two conditions: withdrawal of P alone while E2 is maintained, or withdrawal of both E2 and P⁽⁶³⁾. Furthermore, the administration of the antiprogestin, mifepristone (RU486), is associated with marked endometrial ECM breakdown and excessive menstrual bleeding^{(79, 80, 81}.

U.S. Pat. No. 5,583,128 issued Dec. 10, 1996 to Bhatnagar (Ciba-Geigy Corporation) describes the use of aromatase inhibitors administered on a daily basis to female primates, including humans, to effect reliable contraception without at the same time substantially affecting the menstrual cycle of the female primate.

In EP 0340153A1 aromatase inhibitors as anti-fertility pills, but they are intended to reduce estrogen levels of the female mammal in such a manner that ovulations as well as implantation is suppressed. Again, the administration is on a daily basis.

### SUMMARY OF THE INVENTION

Thus, the present invention provides an emergency contraceptive preparation which comprises at least one aromatase inhibitor wherein the preparation comprises at least one dose for administration on one or more days to a female patient following an unprotected sexual encounter until the establishment or continuation of pregnancy of the patient is prevented.

In another aspect, the invention may include in the preparation one or more additional therapeutic agents selected from progesterones, combinations of estrogens and progesterones, antiprogesterones, selective progesterone receptor modulators, selective estrogen receptor modulators, misoprostol, and methotrexate.

In another aspect, the invention provides a method of emergency contraception for a female patient following an unprotected sexual encounter which comprises administering to the patient at least one dose of a preparation comprising at least one aromatase inhibitor on one or more days following an unprotected sexual encounter until the establishment or continuation of pregnancy in the patient is prevented.

The invention also provides the use of one or more daily doses of an aromatase inhibitor either alone or in combination with a plurality of daily doses of other pharmaceutical agents including hormones.

The invention also provides for the use of one or more daily doses of at least one aromatase inhibitor in amounts effective to reduce serum estrogen levels for preventing the achievement and/or establishment and/or maintenance of pregnancy in females exposed to unprotected intercourse.

Another aspect of the invention comprises the use of an aromatase inhibitor in the preparation of a medicament for use as an emergency contraceptive for a female after an unprotected sexual encounter.

The emergency contraceptive may include one or more progesterones, combinations of estrogens and progesterones, antiprogesterones, selective progesterone receptor modulators, selective estrogen receptor modulators, misoprostol, and methotrexate.

The aromatase inhibitor may be combined with currently available emergency contraceptives which may be selected from levonorgestrel and other progesterone compounds in the usual dosage levels.

The inventors have found that oestrogen levels can be significantly decreased during the administration of an aromatase inhibitor to women in the reproductive age group.

While one aromatase inhibitor is preferred for use in the present invention, combinations of aromatase inhibitors may be used especially those aromatase inhibitors having different half-lives. The aromatase inhibitor is preferably selected from aromatase inhibitors having a half-life of about 8 hours to about 4 days, more preferably from aromatase inhibitors having a half-life of about 2 days. Most beneficial are those aromatase inhibitors selected from non-steroidal and reversible aromatase inhibitors. More detail on the types of aromatase inhibitors that may be used in the methods, uses and preparations of the present invention appears subsequently herein.

The other therapeutic substances may be selected from other currently available emergency contraception medications, for example levonorgestrel and other progesterone compounds and combined estrogen/progesterone preparations.

Other substances include antiprogesterone, SPERMS (selective progesterone receptor modulators) e.g. mifeprostone at doses ranging from about 10 to about 600 mg may also be utilized.

Selective estrogen receptor modulators SERMS: Tamoxifen (about 20 mg) in combination with mifepristone about 10 mg may also be combined. Prostaglandins may also be included.

Misoprostol may also be used in doses ranging from about 50 to about 2000 microgram, single and multiple administrations.

Methotrexate is another suitable choice in amounts ranging from about 25 to about 50 mg/m³, for single and multiple administrations.

The aromatase inhibitors that have been found to be most useful of the commercially available forms are those in oral form. This form offers clear advantages over other forms, including convenience and patient compliance. Preferred aromatase inhibitors of those that are commercially available include anastrozole, letrozole, vorozole and exemestane. Exemestane (Aromasin™) is an example of a steroidal non-reversible aromatase inhibitor that may be used in the present invention.

The daily doses required for the present invention depend on the type of aromatase inhibitor that is used. Some inhibitors are more active than others and hence lower amounts of the former inhibitors could be used.

Typically, the amount of aromatase inhibitor for preventing the achievement and/or establishment and/or maintenance of pregnancy in females exposed to unprotected sexual encounter that may lead to pregnancy may be selected from amounts that lower estrogen levels resulting in disruption of endometrial integrity leading to shedding of the endometrium and induced menstruation or at least destroying the integrity of the endometrial structure that will be unfavorable for the implantation of a fertilized oocyte or maintenance of early pregnancy.

Examples of preferred dosages are as follows. When the aromatase inhibitor is letrozole, it may be administered in a daily dose of from about 2.5 mg to about 60.00 mg. When the aromatase inhibitor is anastrozole, it may be administered in a daily dose of from about 1 mg to about 30 mg. When the aromatase inhibitor is vorazole, the daily dose may be from about 5 to about 100 mg. Exemestane may be administered in a daily dose of about 100 mg to about 2000 mg. 1 to 10 daily doses of the aromatase inhibitor with administration starting on any of days 1 to 10 after exposure to unprotected intercourse, for 1-10 days are constituted to be useful. The daily doses of the aromatase inhibitor comprise five daily doses.

In another form of the invention a single dose of Al is administered in place of the multiple daily doses described above. The aromatase inhibitor is preferably administered in a single dose selected from amounts in the range of from about 5 mg to 60 mg of letrozole or arimidex to about 500 to 2000 mg of exemestane.

The present invention involves the use of at least one aromatase inhibitor, alone or in combination with at least one other therapeutic substance, for emergency contraception i.e. prevention of the establishment and/or continuation of pregnancy following an unprotected sexual encounter that may lead to pregnancy.

The present invention involves several possible mechanisms that can explain the success of aromatase inhibitors in preventing the occurrence and/or establishment and/or the continuation of pregnancy following unprotected sexual encounter.

The main hypothesis is the “induction of menstruation” hypothesis i.e., disruption of the endometrium and its breakdown leading to the induction of menstruation. Endometrial breakdown in the form of menstruation will result in the prevention of the occurrence and/or establishment and/or continuation of pregnancy. This is clearly due to the absence of a receptive endometrium that can support pregnancy.

We propose that the induction of menstruation by aromatase inhibition is the result of two mechanisms: first, a direct mechanism involving local estrogen withdrawal by inhibition of endometrial aromatase and local endometrial estrogen production, and second, by a direct or indirect intraovarian effect resulting from steroid precursor substrate failure, (i.e., androgens and progestins) to be converted to estrogens by reduced aromatase levels induced by the Al resulting in a drop in circulating estrogen levels of

Both “estrogen withdrawal” actions are expected to result in a cascade of events resulting in the disruption of endometrial integrity leading to its breakdown and the induction of menstruation. We believe that endometrial disruption will occur, regardless of the stage of the menstrual cycle in which the aromatase inhibitor is given. Secondly, in cases where Al administration occurs prior to ovulation, we believe that the final stages of folliculogenesis, oocyte/cumulus maturation and ovulation will disrupted effectively obviating conception. In cases where Al is administered following ovulation, Al is expected to prevent luteal estrogen production resulting in diminished luteal function. The combination of these mechanisms is expected to result in markedly improved efficacy of emergency contraception.

We believe that other potential mechanisms may work as well in the prevention of the establishment and/or continuation of pregnancy including but not restricted to the effect of a marked hypoestrogenic milieu on:
Transport, capacitation and interaction of the sperm with the oocyte
Ovulation failure
Luteal function
Maturation, transport and development of the cumulus-oocyte complex
Fallopian tube transportation of the oocyte and/or zygote
Diminished capacity for fertilization
Early development of the embryo
Implantation capacity of the blastocyst

We believe that the major advantage of our invention over any of the currently available methods of emergency contraception is its efficacy irrespective to the interval from the unprotected sexual encounter. We believe that the success of aromatase inhibition in the prevention of the establishment and/or continuation of pregnancy is not limited by the interval between the unprotected sexual encounter and the beginning of aromatase inhibition administration.

The currently available methods of emergency contraception have the major drawback of limited window of use of generally 3 days after the unprotected sexual encounter. Moreover, the failure rates are known to escalate proportionately with the interval between beginning of administration and the unprotected sexual encounter.

Moreover, the lack of immediate availability of the emergency contraceptive method is believed to be the main reason behind the failure of the current emergency contraceptives in preventing unwanted pregnancy and would not be a problem with the current invention because of the longer window of effective use after unprotected sexual encounter.

Other potential advantages include, the extreme safety profile of third generation aromatase inhibitors and their high tolerability. Specifically, the absence of significant nausea and vomiting that limit the success or even the use of the currently available emergency contraceptives in some women is a major advantage.

In addition, the third generation aromatase inhibitors are orally administered, without known significant allergic reactions, drug interactions or contraindications. The cost of aromatase inhibitors is not expected to exceed the currently available methods of emergency contraceptives.

Finally, the rapid onset of menstruation , within days of aromatase inhibitor administration, should provide a rapid indication of success of the therapy leading to lessening patient anxiety about an unwanted pregnancy.

Although aromatase inhibitors have not been used in women of the reproductive age group, we have discovered the effectiveness of these drugs to effectively decrease estrogen levels in women of the reproductive age group. Moreover, we found that estrogen levels following induction or augmentation of ovulation with aromatase inhibitors were significantly lower (especially serum E2 concentration/mature follicle) when compared with conventional stimulation protocols.

Aromatase Inhibitor

By “aromatase inhibitors” there are to be understood substances that inhibit the enzyme aromatase (=oestrogen synthetase), which is responsible for converting androgens to oestrogens.

Aromatase inhibitors may have a non-steroidal or a steroidal chemical structure. According to the present invention, both non-steroidal aromatase inhibitors and steroidal aromatase inhibitors can be used.

By aromatase inhibitors there are to be understood especially those substances that in a determination of the in vitro inhibition of aromatase activity exhibit IC₅₀ values of 10⁻⁵ M or lower, especially 10⁻⁶ M or lower, preferably 10⁻⁷ M or lower and most especially 10⁻⁸ M or lower.

The in vitro inhibition of aromatase activity can be demonstrated, for example, using the methods described in J. Biol. Chem. 249, 5364 (1974) or in J. Enzyme Inhib. 4, 169 (1990). In addition, IC₅₀ values for aromatase inhibition can be obtained, for example, in vitro by a direct product isolation method relating to inhibition of the conversion of 4-¹⁴ C-androstenedione to 4-¹⁴ C-oestrone in human placental microsomes.

By aromatase inhibitors there are to be understood most especially substances for which the minimum effective dose in the case of in vivo aromatase inhibition is 10 mg/kg or less, especially 1 mg/kg or less, preferably 0.1 mg/kg or less and most especially 0.01 mg/kg or less.

In vivo aromatase inhibition can be determined, for example, by the following method [see J. Enzyme Inhib. 4, 179 (1990)]: androstenedione (30 mg/kg subcutaneously) is administered on its own or together with an aromatase inhibitor (orally or subcutaneously) to sexually immature female rats for a period of 4 days. After the fourth administration, the rats are sacrificed and the uteri are isolated and weighed. The aromatase inhibition is determined by the extent to which the hypertrophy of the uterus induced by the administration of androstenedione alone is suppressed or reduced by the simultaneous administration of the aromatase inhibitor.

The following groups of compounds are listed as examples of aromatase inhibitors. Each individual group forms a group of aromatase inhibitors that can be used successfully in accordance with the present invention:
(a) The compounds of formulae I and I* as defined in EP-A-165 904. These are especially the compounds of formula I

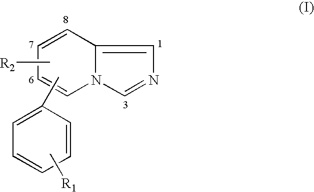

wherein R₁ is hydrogen, lower alkyl; lower alkyl substituted by hydroxy, lower alkoxy, lower alkanoyloxy, lower alkanoyl, amino, lower alkylamino, di-lower alkylamino, halogen, sulfo, carboxy, lower alkoxycarbonyl, carbamoyl or by cyano; nitro, halogen, hydroxy, lower alkoxy, lower alkanoyloxy, phenylsulfonyloxy, lower alkylsulfonyloxy, mercapto, lower alkylthio, lower alkylsulfinyl, lower alkylsulfonyl, lower alkanoylthio, amino, lower alkylamino, di-lower alkylamino, lower alkyleneamino, N-morpholino, N-thiomorpholino, N-piperazino that is unsubstituted or lower alkyl-substituted in the 4-position, tri-lower alkylammonio, sulfo, lower alkoxysulfonyl, sulfamoyl, lower alkylsulfamoyl, di-lower alkylsulfamoyl, formyl; iminomethyl that is unsubstituted or substituted at the nitrogen atom by hydroxy, lower alkoxy, lower alkanoyloxy, lower alkyl, phenyl or by amino; C₂-C₇ alkanoyl, benzoyl, carboxy, lower alkoxycarbonyl, carbamoyl, lower alkylcarbamoyl, di-lower alkylcarbamoyl, cyano, 5-tetrazolyl, unsubstituted or lower alkyl-substituted 4,5-dihydro-2-oxazolyl or hydroxycarbamoyl; and R₂ is hydrogen, lower alkyl, phenyl-lower alkyl, carboxy-lower alkyl, lower alkoxycarbonyl-lower alkyl, halogen, hydroxy, lower alkoxy, lower alkanoyloxy, mercapto, lower alkylthio, phenyl-lower alkylthio, phenylthio, lower alkanoylthio, carboxy, lower alkoxycarbonyl or lower alkanoyl; the 7,8-dihydro derivatives thereof; and the compounds of formula I*

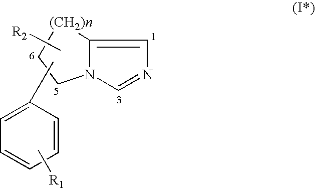

wherein n is 0, 1, 2, 3 or 4; and R₁ and R₂ are as defined above for formula I; it being possible for the phenyl ring in the radicals phenylsulfonyloxy, phenyliminomethyl, benzoyl, phenyl-lower alkyl, phenyl-lower alkylthio and phenylthio to be unsubstituted or substituted by lower alkyl, lower alkoxy or by halogen; it being possible in a compound of formula I* for the two substituents C₆ H₄—R₁ and R₂ to be linked to each of the saturated carbon atoms of the saturated ring, either both to the same carbon atom or both to different carbon atoms, and pharmaceutically acceptable salts thereof.

Individual compounds that may be given special mention here are:

(1) 5-(p-cyanophenyl)imidazo[1,5-a]pyridine,

(2) 5-(p-ethoxycarbonylphenyl)imidazo[1,5-a]pyridine,

(3) 5-(p-carboxyphenyl)imidazo[1,5-a]pyridine,

(4) 5-(p-tert-butylaminocarbonylphenyl)imidazo[1,5-a]pyridine,

(5) 5-(p-ethoxycarbonylphenyl)-5,6,7,8-tetrahydroimidazo[1,5-a]pyridine,

(6) 5-(p-carboxyphenyl)-5,6,7,8-tetrahydroimidazo[1,5-a]pyridine,

(7) 5-(p-carbamoylphenyl)-5,6,7,8-tetrahydroimidazo[1,5-a]pyridine,

(8) 5-(p-tolyl)-5,6,7,8-tetrahydroimidazo[1,5-a]pyridine,

(9) 5-(p-hydroxymethylphenyl)imidazo[1,5-a]pyridine,

(10) 5-(p-cyanophenyl)-7,8-dihydroimidazo[1,5-a]pyridine,

(11) 5-(p-bromophenyl)-5,6,7,8-tetrahydroimidazo[1,5-a]pyridine,

(12) 5-(p-hydroxymethylphenyl)-5,6,7,8-tetrahydroimidazo[1,5-a]pyridine,

(13) 5-(p-formylphenyl)-5,6,7,8-tetrahydroimidazo[1,5-a]pyridine,

(14) 5-(p-cyanophenyl)-5-methylthio-5,6,7,8-tetrahydroimidazo[1,5-a]pyridine,

(15) 5-(p-cyanophenyl)-5-ethoxycarbonyl-5,6,7,8-tetrahydroimidazo[1,5-a]pyridine,

(16) 5-(p-aminophenyl)-5,6,7,8-tetrahydroimidazo[1,5-a]pyridine,

(17) 5-(p-formylphenyl)imidazo[1,5-a]pyridine,

(18) 5-(p-carbamoylphenyl)imidazo[1,5-a]pyridine,

(19) 5H-5-(4-tert-butylaminocarbonylphenyl)-6,7-dihydropyrrolo[1,2-c]imidazole,

(20) 5H-5-(4-cyanophenyl)-6,7-dihydropyrrolo[1,2-c]imidazole,

(21) 5H-5-(4-cyanophenyl)-6,7,8,9-tetrahydroimidazo[1,5-a]azepine,

(22) 5-(4-cyanophenyl)-6-ethoxycarbonylmethyl-5,6,7,8-tetrahydroimidazo[1,5-a]pyridine,

(23) 5-(4-cyanophenyl)-6-carboxymethyl-5,6,7,8-tetrahydroimidazo[1,5-a]pyridine

(24) 5-benzyl-5-(4-cyanophenyl)-5,6,7,8-tetrahydroimidazo[1,5-a]pyridine,

(25) 7-(p-cyanophenyl)-5,6,7,8-tetrahydroimidazo[1,5-a]pyridine,

(26) 7-(p-carbamoylphenyl)-5,6,7,8-tetrahydroimidazo[1,5-a]pyridine,

(27) 5-(p-cyanophenyl)-5,6,7,8-tetrahydroimidazo[1,5-a]pyridine (=Fadrozol).
(b) The compounds of formula I as defined in EP-A 236 940. These are especially the compounds of formula I

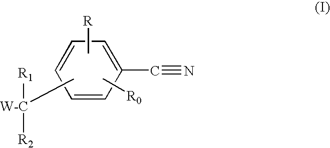

wherein R and R₀, independently of one another, are each hydrogen or lower alkyl, or R and R₀ at adjacent carbon atoms, together with the benzene ting to which they are bonded, form a naphthalene or tetrahydronaphthalene ring; wherein R₁ is hydrogen, lower alkyl, aryl, aryl-lower alkyl or lower alkenyl; R₂ is hydrogen, lower alkyl, aryl, aryl-lower alkyl, (lower alkyl, aryl or aryl-lower alkyl)-thio or lower alkenyl, or wherein R₁ and R₂ together are lower alkylidene or C₄-C₆ alkylene; wherein W is 1-imidazolyl, 1-(1,2,4 or 1,3,4)-triazolyl, 3-pyridyl or one of the mentioned heterocyclic radicals substituted by lower alkyl; and aryl within the context of the above definitions has the following meanings: phenyl that is unsubstituted or substituted by one or two substituents from the group lower alkyl, lower alkoxy, hydroxy, lower alkanoyloxy, nitro, amino, halogen, trifluoromethyl, cyano, carboxy, lower alkoxycarbonyl, carbamoyl, N-lower alkylcarbamoyl, N,N-di-lower alkylcarbamoyl, lower alkanoyl, benzoyl, lower alkylsulfonyl, sulfamoyl, N-lower alkylsulfamoyl and N,N-di-lower alkylsulfamoyl; also thienyl, indolyl, pyridyl or furyl, or one of the four last-mentioned heterocyclic radicals monosubstituted by lower alkyl, lower alkoxy, cyano or by halogen; and pharmaceutically acceptable salts thereof.

Individual compounds from that group that may be given special mention are:

(1) 4-[alpha-(4-cyanophenyl)-1-imidazolylmethyl]-benzonitrile,

(2) 4-[alpha-(3-pyridyl)-1-imidazolylmethyl]-benzonitrile,

(3) 4-[alpha-(4-cyanobenzyl)-1-imidazolylmethyl]-benzonitrile,

(4) 1-(4-cyanophenyl)-1-(1-imidazolyl)-ethylene,

(5) 4-[alpha-(4-cyanophenyl)-1-(1,2,4-triazolyl)methyl]-benzonitrile,

(6) 4-[alpha-(4-cyanophenyl)-3-pyridylmethyl]-benzonitrile.
(c) The compounds of formula I as defined in EP-A-408 509. These are especially the compounds of formula I

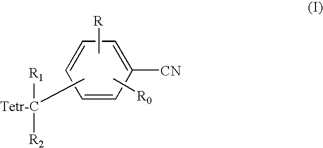

wherein Tetr is 1- or 2-tetrazolyl that is unsubstituted or substituted in the 5-position by lower alkyl, phenyl-lower alkyl or by lower alkanoyl; R and R₂, independently of one another, are each hydrogen; lower alkyl that is unsubstituted or substituted by hydroxy, lower alkoxy, halogen, carboxy, lower alkoxycarbonyl, (amino, lower alkylamino or di-lower alkylamino)-carbonyl or by cyano; lower alkenyl, aryl, heteroaryl, aryl-lower alkyl, C₃-C₆ cycloalkyl, C₃-C₆ cycloalkyl-lower alkyl, lower alkylthio, arylthio or aryl-lower alkylthio; or R₁ and R₂ together are straight-chained C₄-C₆ alkylene that is unsubstituted or substituted by lower alkyl, or are a group —(CH₂)ₘ-1,2-phenylene-(CH₂)ₙ-wherein m and n, independently of one another, are each 1 or 2 and 1,2-phenylene is unsubstituted or substituted in the same way as phenyl in the definition of aryl below, or are lower alkylidene that is unsubstituted or mono- or di-substituted by aryl; and R and R₀, independently of one another, are each hydrogen or lower alkyl; or R and R₀ together, located at adjacent carbon atoms of the benzene ring, are a benzo group that is unsubstituted or substituted in the same way as phenyl in the definition of aryl below; aryl in the above definitions being phenyl that is unsubstituted or substituted by one or more substituents from the group consisting of lower alkyl, lower alkoxy, hydroxy, lower alkanoyloxy, nitro, amino, halogen, trifluoromethyl, carboxy, lower alkoxycarbonyl, (amino, lower alkylamino or di-lower alkylamino)-carbonyl, cyano, lower alkanoyl, benzoyl, lower alkylsulfonyl and (amino, lower alkylamino or di-lower alkylamino)-sulfonyl; heteroaryl in the above definitions being an aromatic heterocyclic radical from the group consisting of pyrrolyl, pyrazolyl, imidazolyl, triazolyl, tetrazolyl, furanyl, thienyl, isoxazolyl, oxazolyl, oxadiazolyl, isothiazolyl, thiazolyl, thiadiazolyl, pyridyl, pyridazinyl, pyrimidyl, pyrazinyl, triazinyl, indolyl, isoindolyl, benzimidazolyl, benzotriazolyl, benzofuranyl, benzothienyl, benzoxazolyl, benzothiazolyl, benzoxadiazolyl, benzothiadiazolyl, quinolyl and isoquinolyl that is unsubstituted or substituted in the same way as phenyl in the definition of aryl above; and pharmaceutically acceptable salts thereof.

Individual compounds from that group that may be given special mention are:

(1) 4-(2-tetrazolyl)methyl-benzonitrile,

(2) 4-[α-(4-cyanophenyl)-(2-tetrazolyl)methyl]-benzonitrile,

(3) 1-cyano-4-(1-tetrazolyl)methyl-naphthalene,

(4) 4-[α-(4-cyanophenyl)-(1-tetrazolyl)methyl]-benzonitrile.
(d) The compounds of formula I as defined in European Patent Application No. 91810110.6. These are especially the compounds of formula I

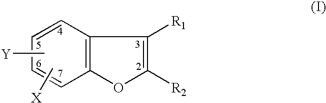

wherein X is halogen, cyano, carbamoyl, N-lower alkylcarbamoyl, N-cycloalkyl-lower alkylcarbamoyl, N,N-di-lower alkylcarbamoyl, N-arylcarbamoyl, hydroxy, lower alkoxy, aryl-lower alkoxy or aryloxy, wherein aryl is phenyl or naphthyl, each of which is unsubstituted or substituted by lower alkyl, hydroxy, lower alkoxy, halogen and/or by trifluoromethyl; Y is a group-CH₂-A wherein A is 1-imidazolyl, 1-(1,2,4-triazolyl), 1-(1,3,4-triazolyl), 1-(1,2,3-triazolyl), 1-tetrazolyl or 2-tetrazolyl, or Y is hydrogen, R₁ and R₁, independently of one another, are each hydrogen, lower alkyl or a group-CH₂-A as defined for Y, or R₁ and R₂ together are —(CH₂)ₙ—wherein n is 3, 4 or 5, with the proviso that one of the radicals Y, R₁ and R₂ is a group-CH₂-A, with the further proviso that in a group-CH₂-A as a meaning of R₁ or R₂, A is other than 1-imidazolyl when X is bromine, cyano or carbamoyl, and with the proviso that in a group-CH_{.2}-A as a meaning of Y, A is other than 1-imidazolyl when X is halogen or lower alkoxy, R₁ is hydrogen and R₂ is hydrogen or lower alkyl, and pharmaceutically acceptable salts thereof.

Individual compounds from that group that may be given special mention are:

(1) 7-cyano-4-[1-(1,2,4-triazolyl)methyl]-2,3-dimethylbenzofuran,

(2) 7-cyano-4-(1-imidazolylmethyl)-2,3-dimethylbenzofuran,

(3) 7-carbamoyl-4-(1-imidazolylmethyl)-2,3-dimethylbenzofuran,

(4) 7-N-(cyclohexylmethyl)carbamoyl-4-(1-imidazolylmethyl)-2,3-dimethylbenzofuran.
(e) The compounds of formula I as defined in Swiss Patent Application 1339/90-7.

These are especially the compounds of formula I

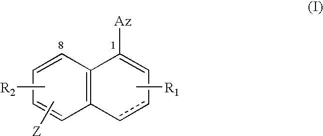

wherein the dotted line denotes an additional bond or no additional bond, Az is imidazolyl, triazolyl or tetrazolyl bonded via a ring nitrogen atom, each of those radicals being unsubstituted or substituted at carbon atoms by lower alkyl or by aryl-lower alkyl, Z is carboxy, lower alkoxycarbonyl, carbamoyl, N-lower alkylcarbamoyl, N,N-di-lower alkylcarbamoyl, N-arylcarbamoyl, cyano, halogen, hydroxy, lower alkoxy, aryl-lower alkoxy, aryloxy, lower alkyl, trifluoromethyl or aryl-lower alkyl, and R₁ and R₂, independently of one another, are each hydrogen, lower alkyl, lower alkoxy, hydroxy, halogen or trifluoromethyl; aryl being phenyl or naphthyl each of which is unsubstituted or substituted by one or two substituents from the group consisting of lower alkyl, lower alkoxy, hydroxy, halogen and trifluoromethyl; with the proviso that neither Z nor R₂ is hydroxy in the 8-position, and pharmaceutically acceptable salts thereof.

Individual compounds from that group that may be given special mention are:

(1) 6-cyano-1-(1-imidazolyl)-3,4-dihydronaphthalene,

(2) 6-cyano-1-[1-(1,2,4-triazolyl)]-3,4-dihydronaphthalene,

(3) 6-chloro-1-(1-imidazolyl)-3,4-dihydronaphthalene,

(4) 6-bromo-1-(1-imidazolyl)-3,4-dihydronaphthalene.
(f) The compounds of formula I as defined in Swiss Patent Application 3014/90-0.

These are especially the compounds of formula I

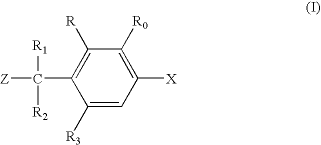

wherein Z is a five-membered nitrogen-containing heteroaromatic ting selected from the group 5-isothiazolyl, 5-thiazolyl, 5-isoxazolyl, 5-oxazolyl, 5-(1,2,3-thiadiazolyl), 5-(1,2,3-oxadiazolyl), 3-(1,2,5-thiadiazolyl), 3-(1,2,5-oxadiazolyl), 4-isothiazolyl, 4-isoxazolyl, 4-(1,2,3-thiadiazol), 4-(1,2,3-oxadiazolyl), 2-(1,3,4-thiadiazolyl), 2-(1,3,4-oxadiazolyl), 5-(1,2,4-thiadiazolyl) and 5-(1,2,4-oxadiazolyl); R and R₀ are hydrogen; or R and R₀ together are a benzo group that is unsubstituted or substituted by lower alkyl, lower alkoxy, hydroxy, halogen or by trifluoromethyl; R₁ is hydrogen, hydroxy, chlorine or fluorine; R₃ is hydrogen; R₂ is hydrogen, lower alkyl or phenyl that is unsubstituted or substituted by lower alkyl, lower alkoxy, hydroxy, halogen, trifluoromethyl or by cyano; or R₁ and R₂ together are methylidene; or R₂ and R₃ together are —(CH₂)₃—; or R₁ and R₂ and R₃ together are a group ═CH—(CH₂)₂— wherein the single bone is linked to the benzene ring; X is cyano; and X may also be halogen when R₂ and R₃ together are —(CH₂)₃— or R₁ and R₁ and R₃ together are a group ═CH—(CH₂)₂—; and pharmaceutically acceptable salts thereof.

Individual compounds from that group that may be given special mention are:

(1) 4-[α-(4-cyanophenyl)-α-hydroxy-5-isothiazolylmethyl]-benzonitrile.

(2) 4-[α-(4-cyanophenyl)-5-isothiazolylmethyl]-benzonitrile,

(3) 4-[α-(4-cyanophenyl)-5-thiazolylmethyl]-benzonitrile,

(4) 1-(4-cyanophenyl)-1-(5-thiazolyl)-ethylene,

(5) 6-cyano-1-(5-isothiazolyl)-3,4-dihydronaphthalene,

(6) 6-cyano-1-(5-thiazolyl)-3,4-dihydronaphthalene.
(g) The compounds of formula VI as defined in Swiss Patent Application 3014/90-0.

These are especially the compounds of formula VI

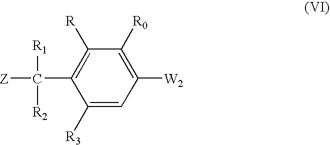

wherein Z is a five-membered nitrogen-containing heteroaromatic ring selected from the group 5-isothiazolyl, 5-thiazolyl, 5-isoxazolyl, 5-oxazolyl, 5-(1,2,3-thiadiazolyl). 5-(1,2,3-oxadiazolyl) 3-(1,2,5-thiadiazolyl), 3-(1,2,5-oxadiazolyl), 4-isothiazolyl. 4-isoxazolyl, 4-(1,2,3-thiadiazolyl), 4-(1,2,3-oxadiazolyl), 2-(1,3,4-thiadiazolyl), 2-(1,3,4-oxadiazolyl), 5-(1,2,4-thiadiazolyl) and 5-(1,2,4-oxadiazolyl); R and R₀ are each hydrogen; or R and R₀ together are a benzo group that is unsubstituted or substituted by lower alkyl, lower alkoxy, hydroxy, halogen or by trifluoromethyl; R₁ is hydrogen, hydroxy, chlorine or fluorine; R₃ is hydrogen; R₂ is hydrogen, lower alkyl or phenyl that is unsubstituted or substituted by lower alkyl, lower alkoxy, hydroxy, halogen, trifluoromethyl, aryl-lower alkoxy or by aryloxy; or R₁ and R₂ together are methylidene, and W₂ is halogen, hydroxy, lower alkoxy, aryl-lower alkoxy or aryloxy; aryl in each case being phenyl that is unsubstituted or substituted by lower alkyl, lower alkoxy, hydroxy, halogen or by trifluoromethyl; and pharmaceutically acceptable salts thereof.

Individual compounds from that group that may be given special mention are:

(1) bis(4,4′-bromophenyl)-(5-isothiazolyl)methanol,

(2) bis(4,4′-bromophenyl)-(5-isothiazolyl)methane,

(3) bis(4,4′-bromophenyl)-(5-thiazolyl)methanol,

(4) bis(4,4′-bromophenyl)-(5-thiazolyl)methane,
(h) The compounds of formula I as defined in Swiss Patent Application 3923/90-4.

These are especially the compounds of formula I

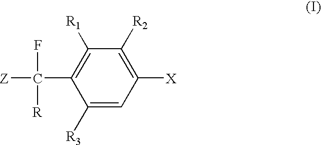

wherein Z is imidazolyl, triazolyl, tetrazolyl, pyrrolyl, pyrazolyl, indolyl, isoindolyl, benzimidazolyl, benzopyrazolyl, benzotriazolyl, pyridyl, pyrimidyl, pyrazinyl, pyridazinyl. triazinyl, quinolinyl or isoquinolinyl, all those radicals being bonded via their heterocyclic rings and all those radicals being unsubstituted or substituted by lower alkyl, hydroxy, lower alkoxy, halogen or by trifluoromethyl: R₁ and R₂, independently of one another, are each hydrogen or lower alkyl; or R₁ and R₂ together are C₃-C₄ alkylene, or a benzo group that is unsubstituted or substituted as indicated below for aryl; R is hydrogen, lower alkyl, aryl or heteroaryl, and X is cyano, carbamoyl, N-lower alkylcarbamoyl, N,N-di-lower alkylcarbamoyl, N,N-lower alkylenecarbamoyl; N,N-lower alkylenecarbamoyl interrupted by-O—, —S-or-NR″—, wherein R″ is hydrogen, lower alkyl or lower alkanoyl; N-cycloalkylcarbamoyl, N-(lower alkyl-substituted cycloalkyl)-carbamoyl, N-cycloalkyl-lower alkylcarbamoyl, N-(lower alkyl-substituted cycloalkyl)-lower alkylcarbamoyl, N-aryl-lower alkylcarbamoyl, N-arylcarbamoyl, N-hydroxycarbamoyl, hydroxy, lower alkoxy, aryl-lower alkoxy or aryloxy; and wherein X is also halogen when Z is imidazolyl, triazolyl, tetrazolyl, pyrrolyl, pyrazolyl, indolyl, isoindolyl, benzimidazolyl, benzopyrazolyl or benzotriazolyl; wherein aryl is phenyl or naphthyl, these radicals being unsubstituted or substituted by from 1 to 4 substituents from the group consisting of lower alkyl, lower alkenyl, lower alkynyl, lower alkylene (linked to two adjacent carbon atoms), C3-C8 cycloalkyl, phenyl-lower alkyl, phenyl; lower alkyl that is substituted in turn by hydroxy, lower alkoxy, phenyl-lower alkoxy, lower alkanoyloxy, halogen, amino, lower alkylamino, di-lower alkylamino, mercapto, lower alkylthio, lower alkylsulfinyl, lower alkylsulfonyl, carboxy, lower alkoxycarbonyl, carbamoyl, N-lower alkylcarbamoyl, N,N-di-lower alkylcarbamoyl and/or by cyano; hydroxy; lower alkoxy, halo-lower alkoxy, phenyl-lower alkoxy, phenoxy, lower alkenyloxy, halo-lower alkenyloxy, lower alkynyloxy, lower alkylenedioxy (linked to two adjacent carbon atoms), lower alkanoyloxy, phenyl-lower alkanoyloxy, phenylcarbonyloxy, mercapto, lower alkylthio, phenyl-lower alkylthio, phenylthio, lower alkylsulfinyl, phenyl-lower alkylsulfinyl, phenylsulfinyl, lower alkylsulfonyl, phenyl-lower alkylsulfonyl, phenylsulfonyl, halogen, nitro, amino, lower alkylamino, C₃-C₈ cycloalkylamino, phenyl-lower alkylamino, phenylamino, di-lower alkylamino, N-lower alkyl-N-phenylamino, N-lower alkyl-N-phenyl-lower alkylamino; lower alkyleneamino or lower alkyleneamino interrupted by-O—, —S-or-NR″- (wherein R″ is hydrogen, lower alkyl or lower alkanoyl); lower alkanoylamino, phenyl-lower alkanoylamino, phenylcarbonylamino, lower alkanoyl, phenyl-lower alkanoyl, phenylcarbonyl, carboxy, lower alkoxycarbonyl, carbamoyl, N-lower alkylcarbamoyl, N,N-di-lower alkylcarbamoyl, N,N-lower alkylenecarbamoyl; N,N-lower alkylenecarbamoyl interrupted by-O—, —S-or-NR″-, wherein R″ is hydrogen, lower alkyl or lower alkanoyl; N-cycloalkylcarbamoyl, N-(lower alkyl-substituted cycloalkyl)-carbamoyl, N-cycloalkyl-lower alkylcarbamoyl, N-(lower alkyl-substituted cycloalkyl)-lower alkylcarbamoyl, N-hydroxycarbamoyl, N-phenyl-lower alkylcarbamoyl, N-phenylcarbamoyl, cyano, sulfo, lower alkoxysulfonyl, sulfamoyl, N-lower alkylsulfamoyl, N,N-di-lower alkylsulfamoyl and N-phenylsulfamoyl; the phenyl groups occurring in the substituents of phenyl and naphthyl in turn being unsubstituted or substituted by lower alkyl, lower alkoxy, hydroxy, halogen and/or by trifluoromethyl; wherein heteroaryl is indolyl, isoindolyl, benzimidazolyl, benzopyrazolyl, benzotriazolyl, benzo[b]furanyl, benzo[b]thienyl, benzoxazolyl or benzothiazolyl, those radicals being unsubstituted or substituted by from 1 to 3 identical or different substituents selected from lower alkyl, hydroxy, lower alkoxy, halogen, cyano and trifluoromethyl; and pharmaceutically acceptable salts thereof.

Those compounds are especially the compounds of formula I whereto Z is
1-imidazolyl, 1-(1,2,4-triazolyl), 1-(1,3,4-triazolyl), 1-(1,2,3-triazolyl), 1-tetrazolyl, 2-tetrazolyl, 3-pyridyl, 4-pyridyl, 4-pyrimidyl, 5-pyrimidinyl or 2-pyrazinyl; R₁ and R₂, independently of one another, are each hydrogen or lower alkyl; or R.₁ and R₂ together are 1,4-butylene or a benzo group; R is lower alkyl; phenyl that is unsubstituted or substituted by cyano, carbamoyl, halogen, lower alkyl, trifluoromethyl, hydroxy, lower alkoxy or by phenoxy; or benzotriazolyl or benzo[b]furanyl, the last two radicals being unsubstituted or substituted by from 1 to 3 identical or different substituents selected from lower alkyl, halogen and cyano; and X is cyano or carbamoyl; and wherein X is also halogen when Z is 1-imidazolyl, 1-(1,2,4-triazolyl), 1-(1,3,4-triazolyl), 1-(1,2,3-triazolyl), 1-tetrazolyl 2-tetrazolyl; and pharmaceutically acceptable salts thereof.

Individual compounds that may be given special mention here are:

(1) 4[α-4-cyanophenyl)-α-fluoro-1-(1,2,4-triazolyl)methyl]-benzonitrile,

(2) 4[α-(4-cyanophenyl)-α-fluoro-(2-tetrazolyl)methyl]-benzonitrile,

(3) 4[α-(4-cyanophenyl)-α-fluoro-(1-tetrazolyl)methyl]-benzonitrile,

(4) 4-[α-(4-cyanophenyl)-α-fluoro-(1-imidazolyl)methyl]-benzonitrile,

(5) 1-methyl-6-[α-(4-chlorophenyl)-α-fluoro-1-(1,2,4-triazolyl)methyl]-benzotriazole,

(6) 4-[α-(4-cyanophenyl)-α-fluoro-1-(1,2,3-triazolyl)methyl]-benzo nitrile,

(7) 7-cyano-4[α-(4-cyanophenyl)-α-fluoro-1-(1,2,4-triazolyl)methyl]-2,3-dimethylbenzo[b]furan,

(8) 4-[α-(4-bromophenyl)-α-fluoro-1-(1,2,4-triazolyl)methyl)-benzo nitrile,

(9) 4[α-(4-cyanophenyl)-α-fluoro-(5-pyrimidyl)methyl]-benzonitrile,

(10) 4[α-(4-bromophenyl)-α-fluoro-(5-pyrimidyl)methyl]-benzonitrile,

(11) 4-[α-(4-cyanophenyl)-α-fluoro-(3-pyridyl)methyl]-benzonitrile,

(12) 7-bromo-4[α-(4-cyanophenyl)-α-fluoro-(1-imidazolyl)methyl]-2,3-dimethylbenzo[b]furan,

(13) 7-bromo-4-[α-(4-cyanophenyl)-α-fluoro-1-(1,2,4-triazolyl)methyl]-2,3-dimethylbenzo[b]furan,

(14) 4-[α-(4-cyanophenyl)-α-fluoro-(5-pyrimidyl)methyl]-benzonitrile,

(15) 4-[α-(4-bromophenyl)-α-fluoro-(5-pyrimidyl)methyl]-benzonitrile,

(16) 4-[α-(4-cyanophenyl)-1-(1,2,3-triazolyl)methyl]-benzonitrile,

(17) 2,3-dimethyl-4-[α-(4-cyanophenyl)-1-(1,2,4-triazolyl)methyl]-7-cyano-benzo[b]furan,

(18) 4-[α-(4-cyanophenyl)-(5-pyrimidyl)methyl]-benzonitrile,

(19) 4-[α-(4-bromophenyl)-(5-pyrimidyl)methyl]-benzonitrile,

(20) 2,3-dimethyl-4-[α-(4-cyanophenyl)-(1-imidazolyl)methyl]-7-bromo-benzo[b]furan,

(21) 2,3-dimethyl-4-[α-(4-cyanophenyl)-1-(1,2,4-triazolyl)methyl]-7-bromo-benzo-[b]furan.
(i) The compounds of formula I as defined in EP-A-114 033. These are especially the compounds of formula I

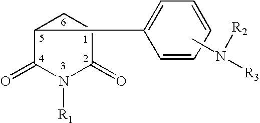

wherein R₁ is hydrogen, R₂ is hydrogen, sulfo, C₁-C₇ alkanoyl or C₁-C₇ alkanesulfonyl and R₃ is hydrogen, or wherein R₁ is C₁-C₁₂ alkyl, C_{.2}-C₁₂ alkenyl, C₂-C₇ alkynyl, C₃-C₁₀ cycloalkyl, C₃-C₁₀ cycloalkenyl, C_{.3} 3-C₆ cycloalkyl-C₁-C₄ alkyl, C₃-C₆ cycloalkyl-C_{.2}-C₄ alkenyl or C₃-C₆ cycloalkenyl-C₁-C₄ alkyl, R₂ is hydrogen, C₁-C₇ alkyl, sulfo, C₁-C₇ alkanoyl or C₁-C₇ alkanesulfonyl and R₃ is hydrogen or C₁-C₇ alkyl, and salts of those compounds.

Individual compounds from that group that may be given special mention are:

(1) 1-(4-aminophenyl)-3-methyl-3-azabicyclo[3.1.0]hexane-2,4-dione,

(2) 1-(4-aminophenyl)-3-n-propyl-3-azabicyclo[3.1.0]hexane-2,4-dione,

(3) 1-(4-aminophenyl)-3-isobutyl-3-azabicyclo[3.1.0]hexane-2,4-dione,

(4) 1-(4-aminophenyl)-3-n-heptyl-3-azabicyclo[3.1.0]hexane-2,4-dione,

(5) 1-(4-aminophenyl)-3-cyclohexylmethyl-3-azabicyclo[3.1.0]hexane-2,4-dione.
(j) The compounds of formula I as defined in EP-A-166 692. These are especially the compounds of formula I

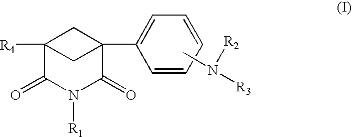

wherein R₁ is hydrogen, alkyl having from 1 to 12 carbon atoms, alkenyl having from 2 to 12 carbon atoms, lower alkynyl, cycloalkyl or cycloalkenyl each having from 3 to 10 carbon atoms, cycloalkyl-lower alkyl having from 4 to 10 carbon atoms, cycloalkyl-lower alkenyl having from 5 to 10 carbon atoms, cycloalkenyl-lower alkyl having from 4 to 10 carbon atoms, or aryl having from 6 to 12 carbon atoms or aryl-lower alkyl having from 7 to 15 carbon atoms, each of which is unsubstituted or substituted by lower alkyl, hydroxy, lower alkoxy, acyloxy, amino, lower alkylamino, di-lower alkylamino, acylamino amino or by halogen, R₂ is hydrogen, lower alkyl, sulfo, lower alkanoyl or lower alkanesulfonyl, sulfonyl, R₃ is hydrogen or lower alkyl and R₄ is hydrogen, lower alkyl, phenyl or phenyl substituted by-N(R₂)(R₃), and salts thereof, radicals described as “lower” containing up to and including 7 carbon atoms.

Individual compounds from that group that may be given special mention are:

(1) 1-(4-aminophenyl)-3-n-propyl-3-azabicyclo[3.1.1]heptane-2,4-dione,

(2) 1-(4-aminophenyl)-3-methyl-3-azabicyclo[3.1.1]heptane-2,4-dione,

(3) 1-(4-aminophenyl)-3-n-decyl-3-azabicyclo[3.1.1]heptane-2,4-dione,

(4) 1-(4-aminophenyl)-3-cyclohexyl-3-azabicyclo[3.1.1]heptane-2,4-dione,

(5) 1-(4-aminophenyl)-3-cyclohexylmethyl-3-azabicyclo[3.1.1]heptane-2,4-dione.
(k) The compounds of formula I as defined in EP-A-356 673. These are especially the compounds of formula I

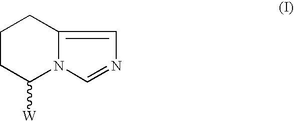

wherein W (a) is a 2-naphthyl or 1-anthryl radical, wherein each benzene ring is unsubstituted or substituted by a substituent selected from halogen, hydroxy, carboxy, cyano and nitro; or (.beta.) is 4-pyridyl, 2-pyrimidyl or 2-pyrazinyl, each of those radicals being unsubstituted or substituted by a substituent selected from halogen, cyano, nitro, C₁-C₄ alkoxy and C₂-C₅ alkoxycarbonyl; and pharmaceutically acceptable salts thereof.

Individual compounds from that group that may be given special mention are:

(1) 5-(2′-naphthyl)-5,6,7,8-tetrahydroimidazo[1,5-a]pyridine,

(2) 5-(4′-pyridyl)-5,6,7,8-tetrahydroimidazo[1,5-a]pyridine.
(I) The compounds of formula I or la as defined in EP-A-337 929. These are especially the compounds of formula I/Ia

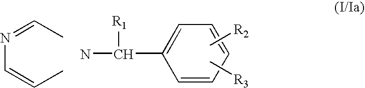

wherein R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl, R₂ is benzyloxy, 3-bromo-, 4-bromo-, 4-chloro-, 2,3-, 2,4-, 4,5- or 4,6-dichloro-benzyloxy, and R₃ is cyano; C₂-C₁₀ alkanoyl that is unsubstituted or mono- or poly-substituted by halogen, methoxy, amino, hydroxy and/or by cyano; benzoyl that is unsubstituted or substituted by one or more substituents from the group halogen, C₁-C₄ alkyl, methoxy, amino, hydroxy and cyano; carboxy, (methoxy, ethoxy or butoxy)-carbonyl, carbamoyl, N-isopropylcarbamoyl, N-phenylcarbamoyl, N-pyrrolidylcarbonyl, nitro or amino; and salts thereof.

Individual compounds from that group that may be given special mention are:

(1) 4-(2,4-dichlorobenzyloxy)-3-[1-(1-imidazolyl)-butyl]-benzonitrile,

(2) (4-(4-bromobenzyloxy)-3-[1-(1-imidazolyl)-butyl]-phenyl pentyl ketone,

(3) 4-(4-bromobenzyloxy)-3-[1-(1-imidazolyl)-butyl]-benzanilide,

(4) 4-(4-bromobenzyloxy)-3-[1-(1-imidazolyl)-butyl]-benzoic acid,

(5) 3-(2,4-dichlorobenzyloxy)-4-[1-(1-imidazolyl)-butyl]-benzonitrile,

(6) 3-(2,4-dichlorobenzyloxy)-4-[1-(1-imidazolyl)-butyl]-benzoic acid methyl ester,

(7) 3-(2,4-dichlorobenzyloxy)-4-[1-(1-imidazolyl)-butyl]-benzoic acid,

(8) 3-(3-bromobenzyloxy)-4-[1-(1-imidazolyl)-butyl]-benzonitrile,

(9) 4-(3-bromobenzyloxy)-3-[1-(1-imidazolyl)-butyl]-benzonitrile,

(10) 3-(4-bromobenzyloxy)-4-[1-(1-imidazolyl)-butyl]-benzoic acid,

(11) 3-(4-bromobenzyloxy)-4-[1-(1-imidazolyl)-butyl]-benzanilide,

(12) 3-(4-bromobenzyloxy)-4-[1-(1-imidazolyl)-butyl]-phenyl pentyl ketone,

(13) 4-(4-bromobenzyloxy)-3-[1-(1-imidazolyl)-butyl]-benzonitrile,

(14) 3-(4-bromobenzyloxy)-4-[1-(1-imidazolyl)-butyl]-benzonitrile,

(15) 4-nitro-2-[1-(1-imidazolyl)-butyl]-phenyl-(2,4-dichlorobenzyl)ether,

(16) 4-amino-2-[1-(1-imidazolyl)-butyl]-phenyl-(2,4-dichlorobenzyl)ether,

(17) (2,4-dichlorobenzyl)-[2-(1-imidazolyl-methyl)-4-nitrophenyl]ether.
(m) The compounds of formula I as defined in EP-A-337 928. These are especially the compounds of formula I

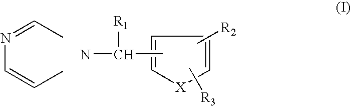

wherein R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl, R₂ is hydrogen, halogen, cyano, methyl, hydroxymethyl, cyanomethyl, methoxymethyl, pyrrolidinylmethyl, carboxy, (methoxy, ethoxy or butoxy)-carbonyl, carbamoyl, N-isopropylcarbamoyl, N-phenylcarbamoyl, N-pyrrolidylcarbonyl; C₂-C₁₀ alkanoyl that is unsubstituted or mono- or poly-substituted by halogen, methoxy, ethoxy, amino, hydroxy and/or by cyano; or benzoyl that is unsubstituted or substituted by one or more substituents from the group halogen, C₁-C₄ alkyl, methoxy, ethoxy, amino, hydroxy and cyano, R₃ is hydrogen, benzyloxy, 3-bromo-, 4-bromo-, 4-chloro-, 2,3-, 2,4-, 4,5- or 4,6-dichlorobenzyloxy, and X is-CH═N—; —CH═N(—O)-or-S—; and salts thereof.

Individual compounds from that group that may be given special mention are:

(1) 5-[1-(1-imidazolyl)-butyl]-thiophene-2-carbonitrile,

(2) 2-[1-(1-imidazolyl)-butyl]-thiophene-4-carbonitrile,

(3) 2-[1-(1-imidazolyl)-butyl]-4-bromo-thiophene,

(4) 2-[1-(1-imidazolyl)-butyl]-5-bromo-thiophene,

(5) 5-[1-(1-imidazolyl)-butyl]-2-thienyl pentyl ketone,

(6) 5-[1-(1-imidazolyl)-butyl]-2-thienyl ethyl ketone,

(7) 5-(4-chlorobenzyloxy)-4-[1-(1-imidazolyl)-pentyl]-pyridine-2-carbonitrile,

(8) 3-(4-chlorobenzyloxy)-4-[1-(1-imidazolyl)-pentyl]-pyridine-2-carbonitrile,

(9) 3-(4-chlorobenzyloxy)-4-[1-(1-imidazolyl)-pentyl]-pyridine-N-oxide,

(10) 3-(4-chlorobenzyloxy)-4-[1-(1-imidazolyl)-pentyl]-pyridine.
(n) The compounds of formula I as defined in EP-A-340 153. These are especially the compounds of formula I

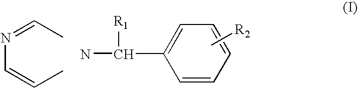

wherein R₁ is hydrogen, methyl, ethyl, propyl, propenyl, isopropyl, butyl, hexyl, octyl, decyl, cyclopentyl, cyclohexyl, cyclopentylmethyl, cyclohexylmethyl or benzyl, and R₂ is a radical from the group methyl, ethyl, propyl, benzyl, phenyl and ethenyl that is substituted by hydroxy, cyano, methoxy, butoxy, phenoxy, amino, pyrrolidinyl, carboxy, lower alkoxycarbonyl or by carbamoyl; or R₂ is formyl or derivatised formyl that can be obtained by reaction of the formyl group with an amine or amine derivative from the group hydroxylamine, O-methylhydroxylamine, O-ethylhydroxylamine, O-allylhydroxylamine, O-benzylhydroxylamine, O-4-nitrobenzyloxyhydroxylamine, O-2,3,4,5,6-pentafluorobenzyloxyhydroxylamine, semicarbazide, thiosemicarbazide, ethylamine and aniline; acetyl, propionyl, butyryl, valeryl, caproyl; benzoyl that is unsubstituted or substituted by one or more substituents from the group halogen, C₁-C₄-alkyl, methoxy, amino, hydroxy and cyano; carboxy, (methoxy, ethoxy or butoxy)carbonyl, carbamoyl, N-isopropylcarbamoyl, N-phenylcarbamoyl or N-pyrrolidylcarbonyl; and salts thereof.

Individual compounds from that group that may be given special mention are:

(1) 4-(1-(1-imidazolyl)-butyl)-benzoic acid methyl ester,

(2) 4-(1-(1-imidazolyl)-butyl)-benzoic acid butyl ester,

(3) 4-(1-(1-imidazolyl)-butyl)-phenyl-acetonitrile,

(4) 4-(1-(1-imidazolyl)-butyl)-benzaldehyde,

(5) 4-(1-(1-imidazolyl)-butyl)-benzyl alcohol,

(6) {4-[1-(1-imidazolyl)-butyl]-phenyl }-2-propyl ketone,

(7) 4-[1-(1-imidazolyl)-butyl]-phenyl propyl ketone,

(8) 4-[1-(1-imidazolyl)-butyl]-phenyl butyl ketone,

(9) 4-[1-(1-imidazolyl)-butyl]-phenyl pentyl ketone,

(10) 4-[1-(1-imidazolyl)-butyl]-phenyl hexyl ketone.
(o) The compounds of formula I as defined in DE-A-4 014 006. These are specially the compounds of formula I

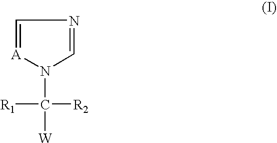

wherein A is an N-atom or a CH radical and W is a radical of the formula

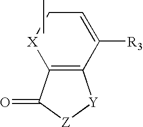

wherein X is an oxygen or a sulfur atom or a-CH═CH-group and Y is a methylene group, an oxygen or a sulfur atom and Z is a —(CH₂)ₙ-group wherein n=1, 2 or 3 and either
a) R₃ in W is a hydrogen atom and R₁ and R₂, independently of one another, are each a hydrogen atom, a C₁- to C₁₀ alkyl group or a C₃- to C₇ cycloalkyl group, or
b) R₂ is as defined under a) and R₁ together with R₃ forms a —(CH₂)ₘ-group wherein m=2, 3, or 4, and their pharmaceutically acceptable addition salts with acids.

Individual compounds from that group that may be given special mention are:

(1) 5-[1-(1-imidazolyl)-butyl]-1-indanone,

(2) 7-[1-(1-imidazolyl)-butyl]-1-indanone,

(3) 6-[1-(1-imidazolyl)-butyl]-1-indanone,

(4) 6-(1-imidazolyl)-6,7,8,9-tetrahydro-1H-benz[e]inden-3(2H)-one,

(5) 2-[1-(1-imidazolyl)-butyl]-4,5-dihydro-6-oxo-cyclopenta[b]-thiophene,

(6) 6-[1-(1-imidazolyl)-butyl]-3,4-dihydro-2H-naphthalen-1-one,

(7) 2-[1-(1-imidazolyl)-butyl]-6,7-dihydro-5H-benzo[b]thiophen-4-one,

(8) 6-[1-(1-imidazolyl)-butyl]-2H-benzo[b]furan-3-one,

(9) 5-[cyclohexyl-(1-imidazolyl)-methyl]-1-indanone,

(10) 2-[1-(1-imidazolyl)-butyl]-4,5-dihydro-6H-benzo[b]thiophen-7-one,

(11) 5-[1-(1-imidazolyl)-1-propyl-butyl]-1-indanone,

(12) 2-[1-(1-imidazolyl)-butyl]-4,5-dihydro-6H-benzo[b]thiophen-7-one,

(13) 2-[1-(1-imidazolyl)-butyl]-4,5-dihydro-6-oxo-cyclopenta[b]-thiophene,

(14) 5-(1-imidazolylmethyl)-1-indanone,

(15) 5-[1-(1,2,4-triazolyl)-methyl]-1-indanone.
(p) The compounds of formula I as disclosed in DE-A-3 926 365. These are especially the compounds of formula I

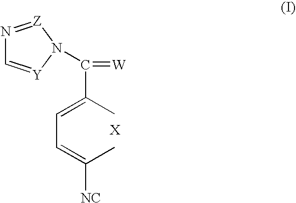

wherein W′ is a cyclopentylidene, cyclohexylidene, cycloheptylidene or 2-adamantylidene radical, X is the grouping-CH═CH—, an oxygen or a sulfur atom, and Y and Z, independently of one another, are each a methine group (CH) or a nitrogen atom, and their pharmaceutically acceptable addition salts with acids.

Individual compounds from that group that may be given special mention are:

(1) 4-[1-cyclohexylidene-1-(imidazolyl)-methyl]-benzonitrile,

(2) 4-[1-cyclopentylidene-1-(imidazolyl)-methyl]-benzonitrile,

(3) 4-[1-cycloheptylidene-1-(imidazolyl)-methyl]-benzonitrile,

(4) 4-[2-adamantylidene-1-(imidazolyl)methyl]-benzonitrile,

(5) 4-[1-cyclohexylidene-1-(1,2,4-triazolyl)-methyl]-benzonitrile,

(6) 4-[1-cyclopentylidene-1-(1,2,4-triazolyl)-methyl]-benzonitrile,

(7) 4-[1-cycloheptylidene-1-(1,2,4-triazolyl)-methyl]-benzonitrile,

(8) 4-[2-adamantylidene-1-(1,2,4-triazolyl)-methyl]-benzonitrile,

(9) 4-[1-cyclohexylidene-1-(1,2,3-triazolyl)-methyl]-benzonitrile,

(10) 4-[1-cyclopentylidene-1-(1,2,3-triazolyl)-methyl]-benzonitrile,

(11) 5-[cyclohexylidene-1-imidazolylmethyl]-thiophene-2-carbonitrile.
(q) The compounds of formula I as defined in DE-A-3 740 125. These are especially the compounds of formula I

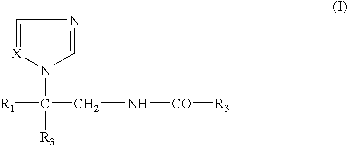

wherein X is CH or N, R₁ and R₂ are identical or different and are each phenyl or halophenyl, and R₃ is C₁-C₄ alkyl; C₁-C₄ alkyl substituted by CN, C₁-C₄ alkoxy, benzyloxy or by C₁-C₄ alkoxy-(mono-, di- or tri-)ethyleneoxy; C₁-C₄ alkoxy, phenyl; phenyl that is substituted by halogen or by cyano; a C₅-C₇ cycloalkyl group that is optionally condensed by benzene, or is thienyl, pyridyl or 2- or 3-indolyl; and acid addition salts thereof.

An individual compound from that group that may be given special mention is:

(1) 2,2-bis(4-chlorophenyl)-2-(1H-imidazol-1-yl)-1-(4-chlorobenzoyl-amino)ethane.
(r) The compounds of formula I as defined in EP-A-293 978. These are especially the compounds of formula I

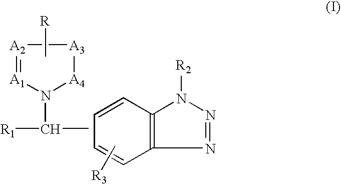

pharmaceutically acceptable salts and stereochemically isomeric forms thereof, wherein-A₁=A₂ -A₃=A₄- is a divalent radical selected from-CH═N—CH═CH—, —CH═N—CH═N-and-CH═N—N═CH—, R is hydrogen or C₁-C₆ alkyl; R₁ is hydrogen, C₁-C₁₀ alkyl, C₃-C₇ cycloalkyl, A₁, Ar₂—C₁′-C₆ alkyl, C₂-C₆ alkenyl or C₂-C₆ alkynyl: R₂ is hydrogen; C₁-C₁₀ alkyl that is unsubstituted or substituted by Ar₁; C₃-C₇ cycloalkyl, hydroxy, C₁-C₆ alkoxy, Ar₁, C₂-C₆ alkenyl, C₂-C₆ alkynyl, C₃-C₇ cycloalkyl, bicyclo[2.2.1]heptan-2-yl, 2,3-dihydro-1H-indenyl, 1,2,3,4-tetrahydronaphthyl, hydroxy; C₂-C₆ alkenyloxy that is unsubstituted or substituted by Ar₂; C₂-C₆ alkynyloxy; pyrimidyloxy; di(Ar₂)methoxy, (1-C₁-C₄ alkyl-4-piperidinyl)oxy, C₁-C₁₀ alkoxy; or C_{C}₁-C₁₀ alkoxy that is substituted by halogen, hydroxy, C₁-C₆ alkyloxy, amino, mono- or di-(C₁-C₆ alkyl)amino, trifluoromethyl, carboxy, C₁-C₆ alkoxycarbonyl, Ar.sub.l, Ar₂—O—, Ar₂—S—, C₃-C₇ cycloalkyl, 2,3-dihydro-1,4-benzodioxinyl, 1H-benzimidazolyl, C₁-C₄ alkyl-substituted 1H-benzimidazolyl, (1,1′-biphenyl)-4-yl or by 2,3-dihydro-2-oxo-1H-benzimidazolyl; and R₃ is hydrogen, nitro, amino, mono- or di-(C₁-C₆ alkyl)amino, halogen, C₁-C₆ alkyl, hydroxy or C₁-C₆ alkoxy; wherein Ar₁ is phenyl, substituted phenyl, naphthyl, pyridyl, aminopyridyl, imidazolyl, triazolyl, thienyl, halothienyl, furanyl, C₁-C₆ alkylfuranyl, halofuranyl or thiazolyl; wherein Ar₂ is phenyl, substituted phenyl or pyridyl; and wherein “substituted phenyl” is phenyl that is substituted by up to 3 substituents in each case selected independently of one another from the group consisting of halogen, hydroxy, hydroxymethyl, trifluoromethyl, C₁-C₆ alkyl, C₁-C₆ alkoxy, C₁-C₆ alkoxycarbonyl, carboxy, formyl, hydroxyiminomethyl, cyano, amino, mono- and di-(C₁-C₆ alkyl)amino and nitro.

Individual compounds from that group that may be given special mention are:

(1) 6-[(1H-imidazol-1-yl)-phenylmethyl]-1-methyl-1H-benzotriazole,

(2) 6-[(4-chlorophenyl)(1H-1,2,4-triazol-1-y1)methyl]-1-methyl-1H-benzotriazole.
(s) The compounds of formula II as defined in EP-A-250 198, especially

(1) 2-(4-chlorophenyl)-1,1-di(1,2,4-triazol-1-ylmethyl)ethanol,

(2) 2-(4-fluorophenyl)-1,1-di(1,2,4-triazol-1-ylmethyl)ethanol,

(3) 2-(2-fluoro-4-trifluoromethylphenyl)-1,1-di(1,2,4-triazol-1-ylmethyl)ethanol,

(4) 2-(2,4-dichlorophenyl)-1,1-di(1,2,4-triazol-1-ylmethyl)ethanol,

(5) 2-(4-chlorophenyl)-1,1-di(1,2,4-triazol-1-ylmethyl)-ethanol,

(6) 2-(4-fluorophenyl)-1,1-di(1,2,4-triazol-1-yl-methyl)ethanol.
(t) The compounds of formula I as defined in EP-A-281 283, especially (1) (1R*2R1-6-fluoro-2-(4-fluorophenyl)-1,2,3,4-tetrahydro-1-(1H-1,2,4methylnaphthalene,

(2) (1R *,2R *)-6-fluoro-2-(4-fluorophenyl)-1,2,3,4-tetrahydro-1-(1H-imidazolylmethyl)-naphthalene,

(3) (1R*,2R*)- and (1R*,2S*)-2-(4-fluorophenyl)-1,2,3,4-tetrahydro-1-(1H-1,2,4-triazol-1-ylmethyl)naphthalene-6-carbonitrile,

(4) (1R*,2R*)- and (1R*,2S*)-2-(4-fluorophenyl)-1,2,3,4-tetrahydro-1-(1H-imidazolylmethyl)naphthalene-6-carbonitrile,

(5) (1R*,2R*)- and (1R*,2S*)-1,2,3,4-tetrahydro-1-(1H-1,2,4-triazol-1-ylmethyl)-naphthalene-2,6-dicarbonitrile,

(6) (1R*,2R*)- and (1R*,2S*)-1,2,3,4-tetrahydro-1-(1H-imidazol-1-ylmethyl)naphthalene-2,6-dicarbonitrile,

(7) (1R*,2S*)-2-(4-fluorophenyl)-1,2,3,4-tetrahydro-1-(5-methyl-1H-imidazolyl-methyl)naphthalene-6-carbonitrile.
(u) The compounds of formula I as defined in EP-A-296 749, especially

(1) 2,2′-[5-(1H-1,2,4-triazol-1-ylmethyl)-1,3-phenylene]di(2-methylpropiononitrile),

(2) 2,2′-[5-(imidazol-1-ylmethyl)-1,3-phenylene]di(2 methylpropiononitrile),

(3) 2-[3-(1-hydroxy-1-methylethyl)-5-(5H-1,2,4-triazol-1-ylmethyl)phenyl]-2-methylpropiononitrile,

(4) 2,2′-[5-dideuterio(1H-1,2,4-triazol-1-yl)methyl-1,3-phenylene]di(2-trideuteriomethyl-3,3,3-trideuteriopropiononitrile),

(5) 2,2′-[5-dideuterio(1H-1,2,4-triazol-1-yl)methyl-3-phenylene]di(2methylpropiononitrile).
(v) The compounds of formula I as defined in EP-A-299 683, especially

(1) (Z)-α-(1,2,4-triazol-1-ylmethyl)stilbene-4,4′-dicarbonitrile,

(2) (Z)-4′-chloro-α-(1,2,4-triazol-1-ylmethyl)stilbene-4-carbonitrile,

(3) (Z)-α-(1,2,4-triazol-1-ylmethyl)-4′-(trifiuoromethyl)stilbene-4-carbonitrile,

(4) (E)-.beta.-fluoro-α-(1,2,4-triazol-1-ylmethyl)stilbene-4,4′-dicarbonitrile,

(5) (Z)-4′-fluoro-α-(imidazol-1-ylmethyl)stilbene-4-carbonitrile,

(6) (Z)-2′,4′-dichloro-a-(imidazol-1-ylmethyl)stilbene-4-carbonitrile,

(7) (Z)-4′-chloro-α-(imidazol-1-ylmethyl)stilbene-4-carbonitrile,

(8) (Z)-α-(imidazol-1-ylmethyl)stilbene-4,4′dicarbonitrile,

(9) (Z)-α-(5-methylimidazol-1-ylmethyl)stilbene-4,4′-dicarbonitrile,

(10) (Z)-2-[2-(4-cyanophenyl)-3-(1,2,4-triazol-1-yl)propenyl]pyridine-5-carbonitrile.
(w) The compounds of formula I as defined in EP-A-299 684, especially

(1) 2-(4-chlorobenzyl)-2-fluoro-1,3-di(1,2,4-triazol-1-yl)propane,

(2) 2-fluoro-2-(2-fluoro-4-chlorobenzyl)-1,3-di(1,2,4-triazol-1-yl)propane,

(3) 2-fluoro-2-(2-fluoro-4-trifluoromethylbenzyl)-1,3-di(1,2,4-triazol-1-yl)propane,

(4) 3-(4-chlorophenyl)-1-(1,2,4-triazol-1-yl)-2-(1,2,4-triazol-1-ylmethyl)

(5) 2-(4-chloro-α-fluorobenzyl)-1,3-di(1,2,4-triazol-1-yl)propan-2-ol,

(6) 2-(4-chlorobenzyl)-1,3-bis(1,2,4-triazol-1-yl)propane,

(7) 4-[2-(4-chlorophenyl)-1,3-di(1,2,4-triazol-1-ylmethyl)ethoxymethyl]-benzonitrile,

(8) 1-(4-fluorobenzyl)-2-(2fluoro-4-trifluoromethylphenyl)-1,3-di(1,2,4-triazol-1-yl),-propan-2-ol,

(9) 2-(4-chlorophenyl)-1-(4-fluorophenoxy)-1,3-di(1,2,4-triazol-1-yl)propan-2-ol,

(10) 1-(4-cyanobenzyl)-2-(2,4-difluorophenyl)-1,3di(1,2,4-triazol-1-yl)propan-2-ol,

(11) 2-(4-chlorophenyl)-1-phenyl-1,3-di(1,2,4-triazol-1-yl)propan-2-ol.
(x) The compounds as defined in claim 1 of EP-A-316 097, especially

(1) 1,1-dimethyl-8-(1H-1,2,4-triazol-1-ylmethyl)-2(1H)-naphtho[2,1-b]furanone,

(2) 1,2-dihydro1,1-dimethyl-2-oxo-8-(1H-1,2,4-triazol-1-ylmethy)naphtho[2,1-b]-furan-7-carbonitrile,

(3) 1,2-dihydro-1,1-dimethyl-2-oxo-8-(1H-1,2,4-triazol-1-ylmethyl)naphtho[2,1-b]-furan-7-carboxamide,

(4) 1,2-dihydro-1,1-dimethyl-2-oxo-8-[di(1H-1,2,4-triazol-1-yl)methyl]naphtho[2,1-b]-furan-7-carbonitrile.
(y) The compounds of formula I as defined in EP-A-354 689, especially

(1) 4-[2-(4-cyanophenyl)-3-(1,2,4-triazol-1-yl)propyl]benzonitrile,

(2) 4-[1-(4-chlorobenzyl)-2-(1,2,4-triazol-1-yl)ethyl]benzonitrile,

(3) 4-[2-(1,2,4-triazol-1-yl)-1-(4-trifluoromethyl]benzyl)ethyl]benzonitrile,

(4) 4-[2-(1,2,4-triazol-1-yl)-1-(4-[trifluoromethoxy]benzyl)ethyl]benzonitrile.
(z) The compounds of formula (1) as defined in EP-A-354 683, especially

(1) 6-[2-(4-cyanophenyl)-3-(1,2,4-triazol-1-yl)-propyl]nicotinonitrile,

(2) 4-[1-(1,2,4-triazol-1-yl-methyl)-2-(5-[trifluoromethyl]pyrid-2-yl)ethyl]benzonitrile.

Examples of steroidal aromatase inhibitors that may be mentioned are:
(aa) The compounds of formula I as defined in EP-A-181 287. These are especially the compounds of formula I

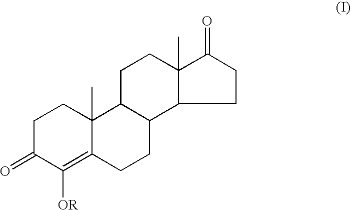

wherein R is hydrogen, acetyl, heptanoyl or benzoyl. An individual compound from that group that may be given special mention is:

(1) 4-hydroxy-4-androstene-3,17-dione.
(ab) The compounds as defined in the claims of U.S. Pat. No. 4,322,416, especially 10-(2-propynyl)-oestr-4-ene-3,17-dione.
(ac) The compounds as defined in the claims of DE-A-3 622 841, especially 6-methyleneandrosta-1,4-diene-3,17-dione.
(ad) The compounds as defined in the claims of GB-A-2 17 1100, especially 4-amino-androsta-1,4,6-triene-3,17-dione.

Also: (ae) androsta-1,4,6-triene-3,17-dione.

The content of the patent applications mentioned under (a) to (z) and (aa) to (ad), especially the subgroups of compounds disclosed therein and the individual compounds disclosed therein as examples, have been incorporated by reference into the disclosure of the present application.

The general terms used hereinbefore and hereinafter to define the compounds have the following meanings:

Organic radicals designated by the term “lower” contain up to and including 7, preferably up to and including 4, carbon atoms.

Acyl is especially lower alkanoyl.

Aryl is, for example, phenyl or 1- or 2-naphthyl, each of which is unsubstituted or substituted by lower alkyl, hydroxy, lower alkoxy, lower alkanoyloxy, amino, lower alkylamino, di-lower alkylamino, lower alkanoylamino or by halogen.

Pharmaceutically acceptable salts of the above-mentioned compounds are, for example, pharmaceutically acceptable acid addition salts or pharmaceutically acceptable metal or ammonium salts.

Pharmaceutically acceptable acid addition salts are especially those with suitable inorganic or organic acids, for example strong mineral acids, such as hydrochloric acid, sulfuric acid or phosphoric acid, or organic acids, especially aliphatic or aromatic carboxylic or sulfonic acids, for example formic, acetic, propionic, succinic, glycolic, lactic, hydroxysuccinic, tartaric, citric, maleic, fumaric, hydroxymaleic, pyruvic, phenylacetic, benzoic, 4-aminobenzoic, anthranilic, 4-hydroxybenzoic, salicylic, 4-aminosalicylic, pamoic, gluconic, nicotinic, methanesulfonic, ethanesulfonic, halobenzenesulfonic, p-toluenesulfonic, naphthalenesulfonic, sulfanilic or cyclohexylsulfamic acid; or with other acidic organic substances, for example ascorbic acid. Pharmaceutically acceptable salts may also be formed, for example, with amino acids, such as arginine or lysine.

Compounds containing acid groups, for example a free carboxy or sulfo group, can also form pharmaceutically acceptable metal or ammonium salts, such as alkali metal or alkaline earth metal salts, for example sodium, potassium, magnesium or calcium salts, also ammonium salts derived from ammonia or suitable organic amines. Them come into consideration especially aliphatic, cycloaliphatic, cycloaliphatic-aliphatic or araliphatic primary, secondary or tertiary mono-, di- or poly-amines, such as lower alkylamines, for example di- or tri-ethylamine, hydroxy-lower alkylamines, for example 2-hydroxyethylamine, bis(2-hydroxyethyl)amine or tris(2-hydroxyethyl)amine, basic aliphatic esters or carboxylic acids, for example 4-aminobenzoic acid 2-diethylaminoethyl ester, lower alkyleneamines, for example 1-ethylpiperidine, cycloalkylamines, for example dicyclohexylamine, benzylamines, for example N,N′-dibenzylethylenediamine; also heterocyclic bases, for example of the pyridine type, for example pyridine, collidine or quinoline. If several acidic or basic groups are present, mono- or poly-salts can be formed. Compounds according to the invention having an acidic and a basic group may also be in the form of internal salts, i.e. in the form of zwitterions and another part of the molecule in the form of a normal salt.

In the case of the above-mentioned individual compounds the pharmaceutically acceptable salts are included in each case insofar as the individual compound is capable of salt formation.

The compounds listed, including the individual compounds mentioned, both in free form and in salt form, may also be in the form of hydrates, or their crystals may include, for example, the solvent used for crystallisation. The present invention relates also to all those forms.

Many of the above-mentioned compounds, including the individual compounds mentioned, contain at least one asymmetric carbon atom. They can therefore occur in the form of R- or S-enantiomers and as enantiomeric mixtures thereof, for example in the form of a racemate. The present invention relates to the use of all those forms and to the use of all further isomers, and of mixtures of at least 2 isomers, for example mixtures of diastereoisomers or enantiomers which can occur when there are one or more further asymmetric centres in the molecule. Also included are, for example, all geometric isomers, for example cis- and trans-isomers, that can occur when the compounds contain one or more double bonds.

Pharmaceutical Formulations

The pharmaceutical compositions that can be prepared according to the invention are compositions for enteral, such as peroral or rectal administration, also for transdermal or sublingual administration, and for parenteral, for example intravenous, subcutaneous and intramuscular, administration. Suitable unit dose forms, especially for peroral and/or sublingual administration, for example dragees, tablets or capsules, comprise preferably from approximately 0.01 mg to approximately 20 mg, especially from approximately 0.1 mg to approximately 10 mg, of one of the above-mentioned compounds or of a pharmaceutically acceptable salt thereof, together with pharmaceutically acceptable carriers. The preferred form of administration is oral. The proportion of active ingredient in such pharmaceutical compositions is generally from approximately 0.001% to approximately 60%, preferably from approximately 0.1% to approximately 20%.

Suitable excipients for pharmaceutical compositions for oral administration are especially fillers, such as sugars, for example lactose, saccharose, mannitol or sorbitol, cellulose preparations and/or calcium phosphates, for example tricalcium phosphate or calcium hydrogen phosphate, and binders, such as starches, for example corn, wheat, rice or potato starch, gelatin, tragacanth, methylcellulose and/or hydroxypropylcellulose, disintegrators, such as the above-mentioned starches, also carboxymethyl starch, cross-linked polyvinylpyrrolidone, agar, alginic acid or a salt thereof, such as sodium alginate, and/or cellulose, for example in the form of crystals, especially in the form of microcrystals, and/or flow regulators and lubricants, for example silicic acid, talc, stearic acid or salts thereof, such as magnesium or calcium stearate, cellulose and/or polyethylene glycol.

Dragee cores can be provided with suitable, optionally enteric, coatings, there being used inter alia concentrated sugar solutions which may comprise gum arabic, talc, polyvinylpyrrolidone, polyethylene glycol and/or titanium dioxide, or coating solutions in suitable solvents or solvent mixtures, or, for the preparation of enteric coatings, solutions of suitable cellulose preparations, such as acetylcellulose phthalate or hydroxypropylmethylcellulose phthalate.

Other orally administrable pharmaceutical compositions are dry-filled capsules consisting of gelatin, and also soft sealed capsules consisting of gelatin and a plasticiser, such as glycerol or sorbitol. The dry-filled capsules may contain the active ingredient in the form of granules, for example in admixture with fillers, such as lactose, binders, such as starches, and/or glidants, such as talc or magnesium stearate, and, if desired, stabilisers. In soft capsules, the active ingredient is preferably dissolved or suspended in suitable oily excipients, such as fatty oils, paraffin oil or liquid polyethylene glycols, to which stabilisers and/or anti-bacterial agents may also be added. There may also be used capsules that are easily bitten through, in order to achieve by means of the sublingual ingestion of the active ingredient that takes place as rapid an action as possible.

Suitable rectally or transvaginally administrable pharmaceutical compositions are, for example, suppositories that consist of a combination of the active ingredient with a suppository base. Suitable suppository bases are, for example, natural or synthetic triglycerides, paraffin hydrocarbons, polyethylene glycols or higher alkanols. There may also be used gelatin rectal capsules, which contain a combination of the active ingredient with a base material. Suitable base materials are, for example, liquid triglycerides, polyethylene glycols or paraffin hydrocarbons.

Suitable formulations for transdermal administration comprise the active ingredient together with a carrier. Advantageous carriers include absorbable pharmacologically acceptable solvents that serve to facilitate the passage through the skin of the host. Transdermal systems are usually in the form of a bandage that comprises a support, a supply container containing the active ingredient, if necessary together with carriers, optionally a separating device that releases the active ingredient onto the skin of the host at a controlled and established rate over a relatively long period of time, and means for securing the system to the skin.

Suitable for parenteral administration are especially aqueous solutions of an active ingredient in water-soluble form, for example in the form of a water-soluble salt, and also suspensions of active ingredient, such as corresponding oily injection suspensions, there being used suitable lipophilic solvents or vehicles, such as fatty oils, for example sesame oil, or synthetic fatty acid esters, for example ethyl oleate, or triglycerides, or aqueous injection suspensions that comprise viscosity-increasing substances, for example sodium carboxymethylcellulose, sorbitol and/or dextran, and, optionally, stabilisers.

Dyes or pigments may be added to the pharmaceutical compositions, especially to the tablets or dragee coatings, for example for identification purposes or to indicate different doses of active ingredient.

The pharmaceutical compositions of the present invention can be prepared in a manner known per se, for example by means of conventional mixing, granulating, confectioning, dissolving or lyophilising processes. For example, pharmaceutical compositions for oral administration can be obtained by combining the active ingredient with solid carriers, optionally granulating a resulting mixture, and processing the mixture or granules, if desired or necessary after the addition of suitable excipients, to form tablets or dragee cores.

### REFERENCES

1. Grimes D A, Raymond E G. Emergency contraception. Ann Intern Med. 2002 Aug. 6; 137(3):180-9.
2. FDA—Food and Drug Administration. (1997). “Prescription Drug Products; Certain Combined Oral Contraceptives for Use as Postcoital Emergency Contraception.” Federal Register, 62(37), 8609-8612.
3. “FDA Approves Progestin-Only Emergency Contraception.” (1999). The Contraception Report, 10(5), 8-10 & 16.
3. Glasier A, Thong K J, Dewar M, Mackie M, Baird D T. Mifepristone (RU 486) compared with high-dose estrogen and progestogen for emergency postcoital contraception. [PMID: 15228399 N Engl J Med. 1992;327:1041-4.
4. Webb A M, Russell J, Elstein M. Comparison of Yuzpe regimen, danazol, and mifepristone (RU486) in oral postcoital contraception. [PMID: 1458074] BMJ. 1992;305:927-31.
5. Trussell J, Rodriguez G, Ellertson C. Updated estimates of the effectiveness of the Yuzpe regimen of emergency contraception. [PMID: 10382076] Contraception. 1999;59:147-51
6. Randomised controlled trial of levonorgestrel versus the Yuzpe regimen of combined oral contraceptives for emergency contraception. Task Force on Postovulatory Methods of Fertility Regulation. [PMID: 9708750] Lancet. 1998;352:428-33.
7. Ho P C, Kwan M S. A prospective randomized comparison of levonorgestrel with the Yuzpe regimen in post-coital contraception. [PMID: 8473453] Hum Reprod. 1993;8:389-92.
8. Piaggio G, von Hertzen H, Grimes D A, Van Look P F. Timing of emergency contraception with levonorgestrel or the Yuzpe regimen. Task Force on Postovulatory Methods of Fertility Regulation [Letter]. [PMID: 10073517] Lancet. 1999;353:721-721.
9. Trussell J, Ellertson C, Rodriguez G. The Yuzpe regimen of emergency contraception: how long after the morning after? [PMID: 8684749] Obstet Gynecol. 1996;88:150-4.
10. Grou F, Rodrigues I. The morning-after pill-how long after? [PMID: 7802062] Am J Obstet Gynecol. 1994;171:1529-34.
11. Ellertson C, Trussell J, Stewart F, Koenig J, Raymond E G, Shochet T. Emergency contraception. [PMID: 11727174] Semin Reprod Med. 2001;19:323-30.
12. Rodrigues I, Grou F, Joly J. Effectiveness of emergency contraceptive pills between 72 and 120 hours after unprotected sexual intercourse. [PMID: 11262449] Am J Obstet Gynecol. 2001;184:531-7
13. Landgren B M, Johannisson E, Aedo A R, Kumar A, Shi Y E. The effect of levonorgestrel administered in large doses at different stages of the cycle on ovarian function and endometrial morphology. [PMID: 2496951] Contraception. 1989;39:275-89.
14. Hapangama D, Glasier A F, Baird D T. The effects of peri-ovulatory administration of levonorgestrel on the menstrual cycle. [PMID: 11368983] Contraception. 2001;63:123-9.
15. Spona J, Matt K, Schneider W H. Study on the action of D-norgestrel as a postcoital contraceptive agent. [PMID: 1116358] Contraception. 1975;11:31-43.
16. Swahn M L, Westlund P, Johannisson E, Bygdeman M. Effect of post-coital contraceptive methods on the endometrium and the menstrual cycle. [PMID: 8906009] Acta Obstet Gynecol Scand. 1996;75:738-44.
17. Ling W Y, Robichaud A, Zayid I, Wrixon W, MacLeod S C. Mode of action of DL-norgestrel and ethinylestradiol combination in postcoital contraception. [PMID: 488410] Fertil Steril. 1979;32:297-302.
18. Rowlands S, Kubba A A, Guillebaud J, Bounds W. A possible mechanism of action of danazol and an ethinylestradiol/norgestrel combination used as postcoital contraceptive agents. [PMID: 3533419] Contraception. 1986;33:539-45.
19. Young D C, Wiehle R D, Joshi S G, Poindexter A N 3rd. Emergency contraception alters progesterone-associated endometrial protein in serum and uterine luminal fluid. [PMID: 8041545] Obstet Gynecol. 1994;84:266-71.
20. Yuzpe A A, Thurlow H J, Ramzy I, Leyshon J I. Post coital contraception-a pilot study. [PMID: 4844513] J Reprod Med. 1974;13:53-8.
21. Kubba A A, White J O, Guillebaud J, Elder M G. The biochemistry of human endometrium after two regimens of postcoital contraception: a dl-norgestrel/ethinylestradiol combination or danazol. [PMID: 3956767] Fertil Steril. 1986;45:512-6.
22. Ling W Y, Wrixon W, Zayid I, Acorn T, Popat R, Wilson E. Mode of action of dl-norgestrel and ethinylestradiol combination in postcoital contraception. II. Effect of postovulatory administration on ovarian function and endometrium. [PMID: 6402387] Fertil Steril. 1983;39:292-7.
23. Taskin O, Brown R W, Young D C, Poindexter A N, Wiehle R D. High doses of oral contraceptives do not alter endometrial alpha 1 and alpha v beta 3 integrins in the late implantation window. [PMID: 7513656] Fertil Steril. 1994;61:850-5.
24. Raymond E G, Lovely L P, Chen-Mok M, Seppälä M, Kurman R J, Lessey B A. Effect of the Yuzpe regimen of emergency contraception on markers of endometrial receptivity. [PMID: 11056131] Hum Reprod. 2000;15:2351-5.
25. Kesserü E, Garmendia F, Westphal N, Parada J. The hormonal and peripheral effects of d-norgestrel in postcoital contraception. [PMID: 4475627] Contraception. 1974;10:411-24.
26. Kesserü E, Camacho-Ortega P, Laudahn G, Schopflin G. In vitro action of progestogens on sperm migration in human cervical mucus. [PMID: 1109940] Fertil Steril. 1975;26:57-61.
27. Ling W Y, Wrixon W, Acorn T, Wilson E, Collins J. Mode of action of dl-norgestrel and ethinylestradiol combination in postcoital contraception. III. Effect of preovulatory administration following the luteinizing hormone surge on ovarian steroidogenesis. [PMID: 6628707] Fertil Steril. 1983;40:631-6.
28. Trussell J, Raymond E G. Statistical evidence about the mechanism of action of the Yuzpe regimen of emergency contraception. [PMID: 10912436] Obstet Gynecol. 1999;93:872-6.
29. Task Force on Postovulatory Methods of Fertility Regulation. Randomised controlled trial of levonorgestrel versus the Yuzpe regimen of combined oral contraceptives for emergency contraception. Lancet 1998;352:428-33.
30. Henshaw S K. Unintended pregnancy in the United States. Fam Plann Perspect 1998;30:24-9, 46.
31. Brown S S, Eisenberg L, eds. The best intentions. Washington, D.C.: National Academy Press, 1995.
32. Trussell J, Stewart F, Guest F, Hatcher R A. Emergency contraceptive pills: A simple proposal to reduce unintended pregnancies. Fam Plann Perspect 1992;24:269-73.
33. AGI—Alan Guttmacher Institute. (2000, accessed 2002, August 9). Facts in Brief: Contraceptive Use: Contraceptive Use [Online
34. Glasier, Anna & David Baird. (1998). “The Effects of Self-Administering Emergency Contraception.” The New England Journal of Medicine, 339(1), 1-4.
35. Piaggio G, von Hertzen H, Grimes D, Van Look P F. Timing of emergency contraception with levonorgestrel or the Yuzpe regimen. Task Force on Postovulatory Methods of Fertility Regulation. Lancet 1999;353:721.
36. Henshaw S K. Abortion incidence and services in the United States, 1995-1996. Fam Plann Perspect 1998;30:263-70, 87.
37. Glasier A. Safety of emergency contraception. J Am Med Womens Assoc 1998;53(5 Suppl 2):219-21.
38. Trussell J, Rodriguez G, Ellertson C. Updated estimates of the effectiveness of the Yuzpe regimen of emergency contraception. Contraception 1999;59:147-51.
39. Espinos J J, Senosiain R, Aura M, Vanrell C, Armengol J, Cuberas N, et al. Safety and effectiveness of hormonal postcoital contraception: A prospective study. Eur J Contracept Reprod Health Care 1999;4:27-33.
40. Vasilakis C, Jick S S, Jick H. The risk of venous thromboembolism in users of postcoital contraceptive pills. Contraception 1999;59:79-83.
41. Creinin M D. A reassessment of efficacy of the Yuzpe regimen of emergency contraception. Hum Reprod 1997;12:496-8.
42. Trussell J, Stewart F, Guest F, Hatcher R A. Emergency contraceptive pills: A simple proposal to reduce unintended pregnancies. Fam Plann Perspect 1992;24:269-73.
43. Delbanco S F, Mauldon J, Smith M D. Little knowledge and limited practice: Emergency contraceptive pills, the public, and the obstetrician-gynecologist. Obstet Gynecol 1997;89:1006-11.
44. Jackson R, Schwarz E, Freedman L, Damey P. Knowledge and willingness to use emergency contraception among low-income post-partum women. Contraception 2000;61:351-7.
45. Raine T, Harper C, Leon K, Damey P. Emergency contraception: Advance provision in a young, high-risk clinic population. Obstet Gynecol 2000;96:1-7.
46. Trussell J, Bull J, Koenig J, Bass M, Allina A, Gamble V N. Call 1-888-NOT-2-LATE: Promoting emergency contraception in the United States. J Am Med Womens Assoc 1998;53(5 Suppl 2):247-50.
47. Delbanco S F, Stewart F H, Koenig J D, Parker M L, Hoff T, McIntosh M. Are we making progress with emergency contraception? Recent findings on American adults and health professionals. J Am Med Womens Assoc 1998;53(5 Suppl 2):242-6.
48. Coeytaux F, Pillsbury B. Bringing emergency contraception to American women: The history and remaining challenges. Womens Health Issues 2001;11:80-6.
49. van Royen A R, Calvin C K, Lightner C R. Knowledge and attitudes about emergency contraception in a military population. Obstet Gynecol 2000;96:921-5.
50. Sills M R, Chamberlain J M, Teach S J. The associations among pediatricians' knowledge, attitudes, and practices regarding emergency contraception. Pediatrics 2000;105:954-6.
51. Sherman C A, Harvey S M, Beckman L J, Petitti D B. Emergency contraception: Knowledge and attitudes of health care providers in a health maintenance organization. Womens Health Issues 2001;11:448-57.
52. Golden N H, Seigel W M, Fisher M, Schneider M, Quijano E, Suss A, et al. Emergency contraception: Pediatricians' knowledge, attitudes, and opinions. Pediatrics 2001;107:287-92.
53. Beckman U, Harvey S M, Sherman C A, Petitti D B. Changes in providers' views and practices about emergency contraception with education. Obstet Gynecol 2001;97:942-6.
54. Trussell J, Duran V, Shochet T, Moore K. Access to emergency contraception. Obstet Gynecol 2000;95:267-70.
55. American College of Obstetricians and Gynecologists. Pharmacists limit women's access to emergency contraception, press release. Washington, D.C.: American College of Obstetricians and Gynecologists, 1999
56. Endres L K, Beshara M, Sondheimer S. Experience with self-administered emergency contraception in a low-income, inner-city family planning program. J Reprod Med 2000;45:827-30.
57. Regulation TFoPMoF. Randomised controlled trial of levonorgestrel versus the Yuzpe regimen of combined oral contraceptives for emergency contraception. Lancet 1998;352:428-33.
58. Piaggio G, von Hertzen H, Grimes D A, Van Look P F. Timing of emergency contraception with levonorgestrel or the Yuzpe regimen. Task Force on Postovulatory Methods of Fertility Regulation. Lancet 1999;353:721.
59. Tabibzadeh S. Decoding implantation and menstruation: the tale of two opposing signals. Front Biosci. 2002 Jun. 1; 7:d1475-86.
60. Tabibzadeh S. The signals and molecular pathways involved in human menstruation, a unique process of tissue destruction and remodelling. Mol Hum Reprod. 1996 February; 2(2):77-92.
61. Noyes, R. W., A. T. Hertig, and J. Rock (1950) Dating the endometrial biopsy. Fertil. Steril. 1: 3-25
62. Markee, J. E. (1940) Menstruation in intraocular transplants in the rhesus monkey. Contributions to Embryology of the Carnegie Institution, 177 211-308
63. Corner, G. W. & Allen, W. M. (1929) Physiology of the corpus luteum. II. Production of a special uterine reaction (progestational proliferation) by extracts of the corpus luteum. American Journal of Physiology, 88 326-339.
64. Fazleabas A T, Strakova Z. Endometrial function: cell specific changes in the uterine environment. Mol Cell Endocrinol 2002; 186:143-147.
65. Tseng L, Mazella J. Endometrial cell specific gene activation during implantation and early pregnancy. Front Biosci 2002; 7:d1566-d1574.
66. Bagchi I C, Li Q, Cheon Y P. Role of steroid-hormone-regulated genes in implantation. Ann NY Acad Sci 2001; 943:68-76.
67. Lessey B A. Adhesion molecules and implantation. J Reprod Immunol 2002; 55:101-112.
68. Ma W, Tan J, Matsumoto H, Robert B, Abrahamson D R, Das S K, Dey S K. Adult tissue angiogenesis: evidence for negative regulation by estrogen in the uterus. Mol-Endocrinol 2001; 15:1983-1992.
69. Fazleabas A T, Bell S C, Fleming S, Sun J, Lessey B A. Distribution of integrins and the extracellular matrix proteins in the baboon endometrium during the menstrual cycle and early pregnancy. Biol Reprod 1997; 56:348-356.
70. Gray C A, Bartol F F, Tarleton B J, Wiley A A, Johnson G A, Bazer F W, Spencer T E. Developmental biology of uterine glands. Biol Reprod 2001; 65:1311-1323.
71. Garcia, E., Bouchard, P., DeBrux, J., Berdah, J., Frydman, R., Schaison, G., Milgrom, E., Perrot-Applanat, M. (1988) Use of immunocytochemistry of progesterone and estrogen receptors for endometrial dating. Journal of Clinical Endocrinology and Metabolism, 67 80-87.
72. Lessey, B. A., Killam, A. P., Metzger, D. A., Haney, A. F., Greene, G. L., McCarty, K. S. (1988) Immunohistochemical analysis of human uterine estrogen and progesterone receptors throughout the menstrual cycle. Journal of Clinical Endocrinology and Metabolism, 67 334-340.
73. Snijders, M. P., de Goeij, A. F. P. M., Debets-Te Baerts, M. J. C., Rousch, M. J., Koudstaal, J., Bosman, F. T. (1992) Immunocytochemical analysis of oestrogen receptors and progesterone receptors in the human uterus throughout the menstrual cycle and after the menopause. Journal of Reproduction and Fertility, 94 363-371.
74. Critchley, H. O. D. & Healy, D. L. (1998) Effects of estrogen and progesterone on the endometrium. In: Estrogens and Progestogens in Clinical Practice (eds I. S. Fraser, R. Jansen, R. Lobo & M. Whitehead), pp. 145-161.Churchill Livingstone, London.
75. Burton, K. A., Hillier, S. G., Habib, F. K., Mason, J. I., Critchley, H. O. D. (2000) Spatial and temporal distribution of the androgen receptor in human endometrium. Journal of Reproduction and Fertility. Abstract. Series 25. Abstract 113.
76. Critchley, H. O. D., Brenner, R. M., Drudy, T. A., Williams, K., Nayak, N. R., Slayden, O. D., Millar, M. R., Saunders, P. T. K. (2001) Estrogen receptor beta, but not estrogen receptor alpha, is present in vascular endothelium of the human and nonhuman primate endometrium. Journal of Clinical Endocrinology and Metabolism, 86 1370-1378.
77. Slayden, O. D., Nayak, N. R., Burton, K. A., Chwalisz, K., Cameron, S. T., Critchley, H. O. D., Baird, D. T., Brenner, R. M. (2001) Progesterone antagonists increase androgen receptor expression in the rhesus macaque and human endometrium. Journal of Clinical Endocrinology and Metabolism, 86 2668-2679.
78. Chauchereau, A., Savouret, J. F., Milgrom, E. (1992) Control of biosynthesis and post-transcriptional modification of progesterone receptor. Biology of Reproduction, 46 174-177.
79. Swahn, M. L., Bygdeman, M., Cekan, S., Xing, S., Masironi, B., Johannisson, E. (1990) The effect of RU486 administered during the early luteal phase on bleeding, hormonal parameters, and endometrium. Human Reproduction, 5 402-408.
80. Spitz, I. M. & Bardin, C. W. (1993) Mifepristone (RU486)—a modulator of progestin and glucocorticoid action. New England Journal of Medicine, 329 404-412.
81. Rudolph-Owen, L. A., Slayden, O., Matrisan, L. M., Brenner, R. M. (1998) Matrix metalloproteinase expression in Macaca mulatta endometrium: evidence for zone-specific regulatory tissue gradients. Biology of Reproduction, 59 1349-1359.

1. An emergency contraceptive preparation which comprises at least one aromatase inhibitor wherein the preparation comprises at least one dose for administration on one or more days to a female patient following an unprotected sexual encounter until the establishment or continuation of pregnancy of the patient is prevented.

2. An emergency contraceptive preparation as claimed in claim 1 wherein the preparation includes one or more additional therapeutic agents selected from progesterones, combinations of estrogens and progesterones, antiprogesterones, selective progesterone receptor modulators, selective estrogen receptor modulators, misoprostol, and methotrexate.

3. An emergency contraceptive preparation as claimed in claim 1 wherein the amount of aromatase inhibitor for administration to the female patient is such that estrogen levels in the patient are lowered until endometrial integrity is disrupted leading to shedding of the endometrium and induced menstruation or at least destroying the integrity of the endometrial structure that will be unfavourable for the implantation of a fertilized oocyte or maintenance of early pregnancy.

4. An emergency contraceptive preparation comprising from 1 to 10 daily doses of the preparation for administration starting on any of days 1 to 10 after exposure to unprotected sexual intercourse for 1 to 10 days.

5. An emergency contraceptive preparation as claimed in claim 4 comprising 5 daily doses administered for a single day on any one of days 1 to 10 after exposure to unprotected sexual intercourse.

6. An emergency contraceptive preparation as claimed in claim 4 wherein the preparation comprises a single dose in an amount selected from about 5 mg to about 60 mg of letrozole, from about 5 mg to about 60 mg of arimidex, from about 100 mg to about 2000 mg of exemestane, from about 5 mg to about 100 mg of vorazole, and from 1 mg to about 30 mg of anastrozole.

7. A method of emergency contraception for a female patient following an unprotected sexual encounter which comprises administering to the patient at least one dose of a preparation comprising at least one aromatase inhibitor on one or more days following an unprotected sexual encounter until the establishment or continuation of pregnancy in the patient is prevented.

8. A method as claimed in claim 7 wherein the at least one dose includes one or more additional therapeutic agents selected from progesterones, combinations of estrogens and progesterones, antiprogesterones, selective progesterone receptor modulators, selective estrogen receptor modulators, misoprostol, and methotrexate.

9. A method as claimed in claim 7 wherein the amount of aromatase inhibitor is selected such that estrogen levels in the patient are lowered until endometrial integrity is disrupted leading to shedding of the endometrium and induced menstruation or at least destroying the integrity of the endometrial structure that will be unfavourable for the implantation of a fertilized oocyte or maintenance of early pregnancy.

10. A method as claimed in claim 9 wherein from 1 to 10 daily doses of the preparation are administered starting on any of days 1 to 10 after exposure to unprotected sexual intercourse for 1 to 10 days.

11. A method as claimed in claim 10 wherein 5 daily doses are administered for a single day on any one of days 1 to 10 after exposure to unprotected sexual intercourse.

12. A method as claimed in claim 10 wherein a single dose in an amount selected from about 5 mg to about 60 mg of letrozole, from about 5 mg to about 60 mg of arimidex, from about 100 mg to about 2000 mg of exemestane, and from about 5 mg to about 100 mg of vorazole is administered.
